(19) United States
(12) Patent Application Publication
(10) Pub. No.: US 2021/0299044 A1
(43) Pub. Date: Sep. 30, 2021

(54) SYSTEMS AND METHODS FOR DELIVERY OF DRUGS AND OTHER SUBSTANCES COMPRISING DEEP EUTECTIC SOLVENTS

(71) Applicant: Novilla Pharmaceuticals, Inc., Arlington, VA (US)

(72) Inventor: Eric T. Fossel, Cambridge, MA (US)

(73) Assignee: Novilla Pharmaceuticals, Inc., Arlington, VA (US)

(21) Appl. No.: 17/264,731

(22) PCT Filed: Jul. 31, 2019
(86) PCT No.: PCT/US2019/044302

§ 371 Date: Jan. 29, 2021

Related U.S. Application Data
(60) Provisional application No. 62/791,110, filed on Jan. 11, 2019.
(60) Provisional application No. 62/778,954, filed on Dec. 13, 2018.
(60) Provisional application No. 62/778,949, filed on Dec. 13, 2018.
(60) Provisional application No. 62/713,022, filed on Aug. 1, 2018.

(51) Int. Cl. A61K 9/08; A61K 9/00; A61K 31/616; A61K 47/18; A61K 31/522; A61K 31/167; A61K 9/107; A61K 9/48; A61K 31/4545; A61K 9/70; A61K 31/407

(52) U.S. Cl. CPC A61K 9/08; A61K 9/0053; A61K 31/616; A61K 47/186; A61K 31/522; A61K 31/407; A61K 9/107; A61K 9/4858; A61K 31/4545; A61K 9/7038; A61K 31/167

(57) ABSTRACT

The present invention generally relates to compositions, including liquid compositions, for the delivery of pharmaceutical agents and other beneficial substances, e.g., in various forms, including but not limited to a liquid, patch, cream, lotion, or gel. For example, some aspects of the present invention are generally directed to formulations comprising deep eutectic solvents. In some cases, such formulations may exhibit surprisingly low melting points, for example, such that the formulations are liquid at ambient temperatures. Such formulations, in some cases, may be useful for facilitating absorption of pharmaceutical agents or beneficial substances that are poorly soluble in water. Also, in certain embodiments, such formulations may be substantially free of water, which some pharmaceutical agents, such as aspirin, can be sensitive to. In addition, in some cases, the formulations can be present in various delivery vehicles, such as patches, creams, lotions, gels, and the like. Such formulations, in some cases, may be useful for containing pharmaceutical agents or beneficial substances that are poorly soluble in water, or are sensitive to water, etc. Accordingly, such formulations may be liquid without necessarily being aqueous, and accordingly can be administered to a subject orally, in liquid form. Other aspects are generally directed to methods of making such compositions, methods of using such compositions, kits including such compositions, etc.

### RELATED APPLICATIONS

This application claims the benefit of U.S. Provisional Patent Application Ser. No. 62/713,022, filed Aug. 1, 2018, entitled “Anhydrous Dosage Forms of Aspirin Based on Deep Eutectic Solvents and Other Specialized Media”; U.S. Provisional Patent Application Ser. No. 62/778,949, filed Dec. 13, 2018, entitled “Systems and Methods for Delivery of Drugs and Other Substances Comprising Deep Eutectic Solvents”; U.S. Provisional Patent Application Ser. No. 62/778,954, filed Dec. 13, 2018, entitled “Methods and Systems Comprising Deep Eutectic Solvents for Poorly Soluble Drugs and Other Applications”; and U.S. Provisional Patent Application Ser. No. 62/791,110, filed Jan. 11, 2019, entitled “Methods and Systems Comprising Deep Eutectic Solvents for Poorly Soluble Drugs and Other Applications.” Each of these is incorporated herein by reference in its entirety.

### FIELD

The present invention generally relates to compositions, including liquid compositions, for the delivery of pharmaceutical agents and other beneficial substances, e.g., in various forms, including but not limited to a liquid, patch, cream, lotion, or gel. For example, in some embodiments, the purpose of the invention is to allow delivery of aspirin to the body in anhydrous dosage forms, including but not limited to liquid, patch, cream, lotion, and gel. Aspirin (acetylsalicylic acid) decomposes on exposure to even the slightest amount of water to acetic acid and salicylic acid. Other pharmaceutical agents and beneficial substances are described in detail below.

### SUMMARY

The present invention generally relates to compositions, including liquid compositions, for the delivery of pharmaceutical agents and other beneficial substances. The subject matter of the present invention involves, in some cases, interrelated products, alternative solutions to a particular problem, and/or a plurality of different uses of one or more systems and/or articles.

For instance, in accordance with some embodiments, there is a need for aspirin to be delivered to the body in a liquid, in a patch, or in a gel, and in other dosage forms that require the drug or pharmaceutical agent to be in solution. Use of deep eutectic solvents such as that formed by urea and choline chloride is able to dissolve aspirin in a substantially anhydrous environment at very high concentrations.

Aspirin dissolved in a pharmaceutically acceptable deep eutectic environment forms the basis of certain embodiments of this invention.

The problems solved by various embodiments of the invention include but are not limited to one or more of the following:

1) An aspirin liquid that can be given to children and others who are not able to (or simply do not want to) take tables or capsules.

2) An aspirin patch which delivers a steady low dose at a constant rate for the use of patients at risk of blood clots and currently take or should take “low dose” oral aspirin.

3) An aspirin cream for the relief of pain which delivers the therapeutic dose to the painful area avoiding exposure to the stomach and providing drastically lower systemic exposure.

4) An aspirin gel which can conveniently be applied to the head for the relief of headache.

These are specific non-limiting examples of various embodiments of the invention, and their uses. Additional embodiments and examples (including other pharmaceutical agents and beneficial substances) are discussed in more detail herein.

In addition, some embodiments of the invention are directed to formulations for facilitating absorption of pharmaceutical agents or beneficial substances that are poorly soluble in water. For example, in certain cases, one or more of these can be contained within a deep eutectic solvent that can be administered, e.g., orally. Also, some embodiments of the invention are directed to formulations that require a substantially anhydrous environment, but can dissolve the aspirin (or other pharmaceutical agents and beneficial substances such as those discussed herein). In contrast, many prior art liquid formulations contain water, and thus are not substantially anhydrous.

In one aspect, the present invention is directed to a composition, such as a composition for oral delivery. In one set of embodiments, the composition comprises 5 mol % to 20 mol % of one or more pharmaceutical agents comprising aspirin, 5 mol % to 95 mol % choline chloride, 5 mol % to 95 mol % urea, and optionally less than 1 mol % water. In some cases, the composition is a liquid. In certain embodiments, the percentages of the pharmaceutical agents, choline chloride, urea, and water sum to at least 80 mol %.

In another set of embodiments, the composition comprises 5 mol % to 20 mol % of one or more pharmaceutical agents and/or beneficial substances, 5 mol % to 95 mol % choline chloride, 5 mol % to 95 mol % urea, and optionally less than 1 mol % water. In some cases, the composition is a liquid. In certain embodiments, the percentages of the pharmaceutical agents, the beneficial substances, choline chloride, urea, and water sum to at least 80 mol %.

In yet another set of embodiments, the composition comprises a deep eutectic solvent, one or more pharmaceutical agents and/or beneficial substances, and optionally less than 1 mol % water. In some cases, the composition is a liquid. In certain embodiments, the percentages of the pharmaceutical agents, the beneficial substances, the deep eutectic solvent, and water sum to at least 80 mol %.

The present invention, in another set of embodiments, is directed to a method, for example, a method of orally administering a composition to a subject. In one set of embodiments, the composition comprises 5 mol % to 20 mol % of one or more pharmaceutical agents comprising aspirin, 5 mol % to 95 mol % choline chloride, 5 mol % to 95 mol % urea, and optionally less than 1 mol % water. In some cases, the composition is a liquid. In certain embodiments, the percentages of pharmaceutical agents, choline chloride, urea, and water sum to at least 80 mol %.

In yet another set of embodiments, the composition comprises 5 mol % to 20 mol % of one or more pharmaceutical agents and/or beneficial substances, 5 mol % to 95 mol % choline chloride, 5 mol % to 95 mol % urea, and optionally less than 1 mol % water. In some cases, the composition is a liquid. In certain embodiments, the percentages of the pharmaceutical agents, the beneficial substances, choline chloride, urea, and water sum to at least 80 mol %. According to still another set of embodiments, the composition comprises a deep eutectic solvent, one or more pharmaceutical agents and/or beneficial substances, and optionally less than 1 mol % water. In some cases, the composition is a liquid. In certain embodiments, the percentages of the pharmaceutical agents, the beneficial substances, the deep eutectic solvent, and water sum to at least 80 mol %.

Additionally, certain embodiments are directed to formulations for facilitating absorption of pharmaceutical agents or beneficial substances that are poorly soluble in water. For example, in certain cases, one or more of these can be contained within a deep eutectic solvent that can used to prepare a delivery vehicle, such as a patch, cream, lotion, or gel.

In one aspect, the present invention is generally directed to a transdermal patch. In one set of embodiments, the transdermal patch comprises a backing layer, and an adhesive comprising choline chloride, urea, one or more pharmaceutical agents, and optionally less than 1 mol % water.

In another aspect, the present invention is generally directed to a composition. In one set of embodiments, the composition comprises an emulsion comprising a first phase and a second phase substantially immiscible in the first phase. In some cases, the first phase comprises choline chloride, urea, one or more pharmaceutical agents, and optionally less than 1 mol % water.

In another set of embodiments, the composition comprises an emulsion comprising a first phase and a second phase substantially immiscible in the first phase, wherein the first phase comprises choline chloride, urea, and loratadine. In some cases, the composition also comprises less than 1 mol % water.

In another set of embodiments, the composition comprises an emulsion comprising a first phase and a second phase substantially immiscible in the first phase, wherein the first phase comprises choline chloride, urea, and ketorolac. In some cases, the composition also comprises less than 1 mol % water.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention encompasses methods of making one or more of the embodiments described herein, for example, a liquid formulation. In still another aspect, the present invention encompasses methods of using one or more of the embodiments described herein, for example, a liquid formulation.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a schematic eutectic diagram in accordance with certain embodiments of the invention.

### DETAILED DESCRIPTION

The present invention generally relates to compositions, including liquid compositions, for the delivery of pharmaceutical agents and other beneficial substances, e.g., in various forms, including but not limited to a liquid, patch, cream, lotion, or gel. For example, some aspects of the present invention are generally directed to formulations comprising deep eutectic solvents. In some cases, such formulations may exhibit surprisingly low melting points, for example, such that the formulations are liquid at ambient temperatures. Such formulations, in some cases, may be useful for facilitating absorption of pharmaceutical agents or beneficial substances that are poorly soluble in water. Also, in certain embodiments, such formulations may be substantially free of water, which some pharmaceutical agents, such as aspirin, can be sensitive to. In addition, in some cases, the formulations can be present in various delivery vehicles, such as patches, creams, lotions, gels, and the like. Such formulations, in some cases, may be useful for containing pharmaceutical agents or beneficial substances that are poorly soluble in water, or are sensitive to water, etc. Accordingly, such formulations may be liquid without necessarily being aqueous, and accordingly can be administered to a subject orally, in liquid form. Other aspects are generally directed to methods of making such compositions, methods of using such compositions, kits including such compositions, etc.

The invention, in some embodiments, is based on aspirin dissolved in a deep eutectic solvent and a combination of other components to produce an anhydrous liquid, patch, cream, or gel. Alternative embodiments include other dosage forms including nasal spray, inhalation form, and other forms which are based on and anhydrous environment for aspirin. Other embodiments include those discussed herein.

For example, one aspect of the invention is generally directed to compositions, including liquid compositions, that are substantially free of water. For instance, the composition may contain less than 1 mol %, less than 0.1 mol %, less than 0.01 mol %, or an undetectable amount of water. The composition may contain a drug or a pharmaceutical agent, for instance, one that can react with water. An example is aspirin (acetylsalicylic acid), which may react water and hydrolyze to acetic acid and salicylic acid; other examples are discussed below. In some cases, the liquid may define a pharmaceutically acceptable carrier, e.g., one that can be administered to a subject (e.g., orally) without deleterious effects. In some cases, more than one pharmaceutically acceptable material may be present within the liquid or delivery vehicle.

In certain embodiments, two or more materials may be present within the composition that each have a melting point, but when mixed together, the resulting mixture may have a melting point that is lower than each of its component materials. Such a phenomenon is commonly referred to as a eutectic mixture. In some cases, the melting point of the mixture may be lower than the melting points of the component materials. For example, the melting point may decrease by at least 10° C., at least 25° C., or at least 50° C. from the lowest of the component melting points. In some cases, the materials and their ratios are chosen such that the mixture is a liquid at room temperature, e.g., the mixture may have a melting point of less than 25° C., such that at ambient temperatures, the mixture is at a temperature above its melting point, and accordingly is liquid. In addition, in some cases, the mixture may be chosen such that it is a liquid at various temperatures, e.g., less than 20° C., less than 10° C., etc. However, it should be understood that the mixture may not necessarily be liquid at room temperature. For instance, in some cases, the mixture may have a melting point of less than 60° C., less than 55° C., less than 50° C., less than 45° C., less than 40° C., less than 35° C., less than 30° C., etc., but greater than room temperature (about 25° C.).

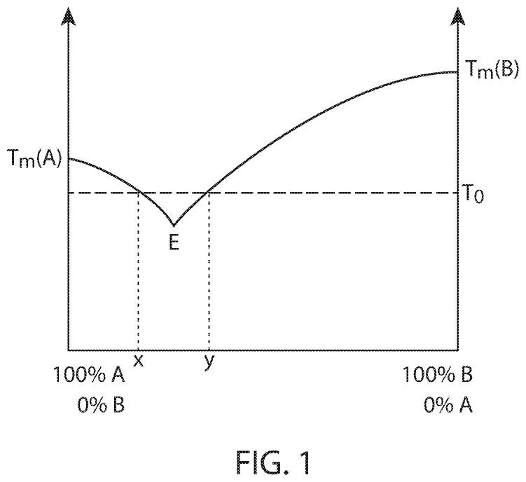

Although such a mixture of two or more materials may exhibit a lowest possible melting point at a specific ratio of materials (commonly referred to as the eutectic point or the eutectic ratio), such as is shown in FIG. 1, it should be understood that the invention is not limited to only those eutectic points or ratios, but instead also encompasses any mixture in which the melting point of the mixture is lower than each of its component materials, and typically where the mixture is liquid at ambient temperatures.

In some cases, the mixture may be a deep eutectic mixture, which can be formed from a mixture of Lewis or Bronsted acids and bases. One example is choline chloride and urea. These can be mixed in a mole ratio of 1:2 to produce a eutectic mixture with a melting point of 12° C. However, as noted above, in other embodiments, other mole ratios may also be used to produce mixtures having lowered melting points, e.g., that are less than ambient temperatures. In addition, other examples of eutectic mixtures are discussed in more detail herein.

Accordingly, in one aspect, the present invention is generally directed to compositions, including liquid compositions. Other delivery vehicle compositions are also contemplated in other embodiments, such as patches, creams, lotions, gels, or the like. In some cases, the composition comprises or consists essentially of a eutectic mixture, e.g., one that exhibits a lower melting point than the components forming the eutectic mixture. Two, three, four, or more materials may be present that can be mixed together to form the eutectic mixture. In some cases, the materials (when separate) are generally solid at ambient temperatures, but form a liquid when mixed together to form the eutectic mixture.

In certain embodiments, two or more materials may be present within the eutectic mixture that each have a melting point, but when mixed together, the resulting mixture may have a melting point that is lower than each of its component materials. In some cases, the difference in melting point may be very large. For example, in some embodiments, the eutectic may exhibit a decrease by at least 10° C., at least 15° C., at least 20° C., at least 25° C., at least 30° C., at least 40° C., at least 50° C., at least 60° C., at least 70° C., at least 80° C., at least 90° C., or ° C., at least 100° C. In addition, in some embodiments, the melting point may be decreased sufficiently so that the eutectic is a liquid at room temperature, e.g., below 25° C. or 20° C. In certain embodiments, the eutectic may be liquid at temperatures of below 15° C., 10° C., 5° C., or 0° C.

One example of a eutectic mixture is urea and choline chloride. Other examples of eutectic mixture include, but are not limited to, phenol/menthol, phenol/choline chloride, phenol/choline chloride/urea, betaine hydrochloride/urea, resorcinol/choline chloride, BHT/choline chloride, chloroxylenol/choline chloride/menthol, choline chloride/citric acid, choline chloride/arginine/urea, choline chloride/niacinamide/urea, camphor/menthol, camphor/menthol/lauryl alcohol, camphor/glycerin/monolaurate/menthol, etc. Additional non-limiting examples include EtNH₃Cl/CF₃CONH₂, EtNH₃Cl/Acetamide, EtNH₃Cl/Urea, ChCl/CF₃CONH₂, AcChCl/Urea, ZnCl₂/urea, ZnCl₂/acetamide, ZnCl₂/ethylene glycol, ZnCl₂/hexanediol, ChCl/glycerol, ChCl/ethylene glycol, ChCl/malonic acid, Et₂(EtOH)NCl/glycerol, Et₂(EtOH)NCl/ethylene glycol, Me(PH)₃PBr/glycerol, Me(PH)₃PBr/ethylene glycol, ChCl/glucose, ChCl/1,4-butanediol, ChCl/CF₃CONH₂, ChCl/imidazole, ChCl/ZnCl₂, ChCl/xylitol, ChCl/sorbitol, ChCl/malonic acid, Bu₄NBr/imidazole, etc. Many of these materials are readily available commercially, and can be mixed together in any suitable ratio.

According to some embodiments, the components of the eutectic mixture may be pharmaceutically acceptable, or are generally recognized as safe (for example, the components may be GRAS components as defined by the US FDA). In some cases, a pharmaceutically acceptable component is one that is generally safe, non-toxic and does not produce harmful or deleterious biological effects (e.g., at doses or amounts comparable to those that would expect typically given to a subject). This may include components acceptable for human or animal use. One example of a pharmaceutically acceptable eutectic mixture is urea/choline chloride. Other non-limiting examples include choline chloride/arginine/urea, camphor/menthol, camphor/menthol/lauryl alcohol, ChCl/glycerol, and others including some of those described above.

Thus, it is important to note that certain embodiments of the invention are generally directed to eutectic mixtures containing non-toxic ingredients, for example, that can safely be ingested, such as a eutectic mixture of urea and choline chloride. Ingesting orally, or applying to the skin, of a subject such non-toxic eutectic mixtures may not substantially deleteriously affect the subject, and accordingly can be used to deliver pharmaceutical agents or other beneficial substances, such as aspirin and other agents described herein.

Accordingly, certain embodiments of the invention are generally directed to systems and methods for facilitating the absorption of pharmaceutical agents or beneficial substances that are poorly soluble in water. For example, a pharmaceutical agent or a beneficial substance may be contained within a eutectic mixture as described herein. In some cases, the eutectic mixture may not necessarily have a significant amount of water, e.g., the eutectic mixture may be substantially anhydrous, or have percentages of water such as those described herein. Accordingly, the solubility of the pharmaceutical agent or a beneficial substance in water is less of an issue in the eutectic mixture, e.g., the pharmaceutical agent or a beneficial substance may have a solubility within the eutectic mixture that is substantially different from its solubility in water. Thus, in certain cases, such eutectic mixtures may be used to deliver pharmaceutical agents or beneficial substances to a subject, e.g., orally, or other techniques such as those described herein, without necessarily being limited to their water solubilities. Accordingly, even poorly soluble pharmaceutical agents or beneficial substances can be effectively administered.

It should be understood, as noted above, that a eutectic mixture need not have ratios of its component materials that produces the lowest possible melting point. Thus, the present invention is not limited to only eutectic ratios of components, but also includes, in other embodiments, other ratios able to cause decreases in the melting point. For example, with reference to FIG. 1 as a non-limiting schematic illustration, two components (“A”) and (“B”) may each exhibit certain melting points in isolation, but when A and B are mixed in various ratios (extending from 100% A on the left to 100% B on the right), the melting point of the components may decrease, e.g., to a point (the eutectic point, E) that is lower than the component melting points of A and B (Tₘ(A) and Tₘ(B). However, it should be understood that the invention is not limited to only that particular ratio of A and B which produces the lowest possible melting point, but also includes other ratios of A and B as well. For instance, a variety of different ratios of A and B may still produce a lower melting point than either A or B separately, even if other ratios of A and B may produce even lower melting point than that. For example, if melting points below some target temperature (To) is desired (e.g., ambient temperature), then any ratios of A and B between x and y would be suitable, not just the ratio at the eutectic point E. It should also be understood that FIG. 1 illustrates an idealized eutectic phase diagram (i.e., it is not to scale), and different eutectic components may exhibit different eutectic behavior, including more complex behaviors than is shown here.

In the eutectics mixtures disclosed above, and other eutectics mixtures described herein, each of the components may be present in any of a wide variety of ratios, e.g., such that the mixture exhibits a lower melting point than the components forming the mixture. In some cases, a first component (e.g., urea) may be present at between 5 mol % and 95 mol %, and the second component (e.g., choline chloride) may be present at between 5 mol % and 95 mol %. A third component (if present) may also be present at between 5 mol % and 95 mol %.

For example, a component may be present within the mixture at at least 5 mol %, at least 10 mol %, at least 15 mol %, at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, etc., and/or at no more than 95 mol %, no more than 90 mol %, no more than 85 mol %, no more than 80 mol %, no more than 75 mol %, no more than 70 mol %, no more than 65 mol %, no more than 60 mol %, no more than 55 mol %, no more than 50 mol %, no more than 45 mol %, no more than 40 mol %, no more than 35 mol %, no more than 30 mol %, no more than 25 mol %, no more than 20 mol %, no more than 15 mol %, no more than 10 mol %, etc. Combinations of any of these are also possible, e.g., a first component may be present at between 30 mol % and 40 mol %, between 25 mol % and 70 mol %, between 40 mol % and 60 mol %, between 60 mol % and 70 mol %, between 45 mol % and 55 mol %, etc. As other non-limiting examples, in some cases, the first component may each be present at between 25 mol % and 45 mol %, between 35 mol % and 45 mol %, between 30 mol % and 40 mol %, etc., and the second component may be present in these percentages, or in a different percentages.

However, it should be understood that in the eutectic mixture, these may sum to 100 mol %, or to other percentages in some cases, e.g., if other materials (such as pharmaceutical agents or beneficial substances) are also present. Thus, for example, the percentages of the first, second, third, etc., components may sum to at least 70 mol %, at least 75 mol %, at least 80 mol %, at least 85 mol %, at least 90 mol %, at least 95 mol %, at least 97 mol %, or at least 99 mol % of the eutectic mixture, depending on other materials that might also be present.

In one set of embodiments, the first components and second components may be present in a mass ratio of between 2:1 and 1:2. For example, the ratio between the first component and the second component may be between 1.5:1 and 1:1.5, or between 1.2:1 and 1:1.2. In some cases, the mass ratio may be at least 1:2, at least 1:1.5, at least 1:1, at least 1.5:1, or at least 2:1, and/or no more than 2:1, no more than 1.5:1, no more than 1:1, no more than 1:1.5, or no more than 2:1. Of course, it should be understood that ratios outside these ranges are also possible in certain embodiments.

As mentioned, in some cases, the composition may be substantially anhydrous. For example, the composition may contain less than 5%, less than 3%, less than 2%, less than 1%, less than 0.5%, less than 0.3%, less than 0.1%, less than 0.05%, less than 0.03%, less than 0.01%, less than 0.005%, less than 0.003%, or less than 0.001% water (by mole). In some cases, no detectable amounts of water may be present.

In some cases, the amount of water present within the composition may be lowered, e.g., such that the composition is substantially anhydrous. For example, the composition, or one or more components forming the composition, may be treated to remove at least some water. For instance, the composition or a component, may be heated, exposed to a desiccant, or chemically reacted to remove water, etc. Other techniques include, but are not limited to, membranes or reverse osmosis, molecular sieves, or fractional freezing. As a specific non-limiting example, the composition, or a component thereof, may be heated to temperatures of at least 40° C., at least 50° C., at least 55° C., at least 60° C., at least 65° C., at least 70° C., at least 75° C., at least 80° C., at least 85° C., at least 90° C., at least 95° C., at least 100° C., etc. to remove water.

Different methods may be used to determine amount of water present, for example, FTIR, IR absorption, electrical resistivity measurements, or the like. As another example, in some embodiments, water may be detected by exposing the composition to a hydrolyzable agent (e.g., aspirin), and determining if the agent is hydrolyzed within the composition after a certain period of time (e.g., a day or a week).

In some cases, a composition has a relatively low viscosity (e.g., comparable to water), although in some cases, the viscosity may be greater (e.g., comparable to honey). In certain embodiments, the viscosity of the composition may be less than 10,000 cP, less than 3,000 cP, less than 1,000 cP, less than 300 cP, less than 100 cP, less than 30 cP, less than 10, less than 3 cP, or less than 1 cP.

As mentioned, other components may be added to the eutectic mixture to form the liquid formulation or delivery vehicle. In certain embodiments, the components may include one or more pharmaceutical agents. One example of a pharmaceutical agent is aspirin (acetylsalicylic acid). Other non-limiting examples include caffeine, acetaminophen (or APAP), niacinamide, naproxen, pseudoephedrine or other decongestants, phenethylamines, amphetamines, or the like. The formulation may comprise one, two, three, or more pharmaceutical agents. For instance, the formulation may comprise only aspirin; aspirin and caffeine; aspirin, caffeine, and acetaminophen; caffeine and niacinamide, or the like. In some cases, a pharmaceutical agent may be present as a salt.

As another example, the component may include a beneficial substance. Non-limiting examples of beneficial substances include vitamins, cofactors, cosmetics, herbs, vitamins, minerals, dietary supplements, peptides, or the like. Non-limiting examples include coenzyme Q₁₀, NAD (nicotinamide adenine dinucleotide), vitamin A, vitamin D, niacin, riboflavin, collagen, or the like. It should be understood that in some cases, there is not a sharp distinction between a pharmaceutical agent and a beneficial substance, e.g., a pharmaceutical agent can also be a beneficial substance, e.g., a substance that is beneficial to the subject. Accordingly, in the descriptions herein, it should be understood that discussions of pharmaceutical agents are by way of example only, and in another embodiment, another beneficial substance may be present instead of (or in addition to) a pharmaceutical agent. In one set of embodiments, the pharmaceutical agent or beneficial substance may be sensitive to water. For instance, a pharmaceutical agent or a beneficial substance may decompose or hydrolyze upon reaction with water. Without wishing to be bound by any theory, it is believed that in certain embodiments, such pharmaceutical agents or beneficial substances may be advantageously contained within a mixture in the invention, e.g., one that is substantially free of water. In some cases, the mixture may be a eutectic mixture, and in certain embodiments, one that is liquid at ambient temperatures. Such compositions may thus limit the amount of exposure of the pharmaceutical agents or beneficial substances to water.

In another set of embodiments, the pharmaceutical agents or beneficial substances may exhibit relative low solubilities in water. Without wishing to be bound by any theory, in some embodiments, such pharmaceutical agents or beneficial substances may be more soluble in mixtures such as those described herein. For instance, a pharmaceutical agent or a beneficial substance may be contained within a mixture that is substantially free of water. In some cases, the mixture may be a eutectic mixture, and in certain embodiments, one that is liquid at ambient temperatures. As examples, in certain embodiments, a pharmaceutical agent or a beneficial substance may have a solubility to water of less than 1000 mg/l, less than 500 mg/l, less than 300 mg/l, less than 100 mg/l, less than 50 mg/l, less than 30 mg/1, or less than 10 mg/1, etc.

Accordingly, some embodiments of the invention may be particularly advantages for the delivery of certain pharmaceutical agents, such as loratadine, that are insoluble or poorly soluble in water (i.e., aqueous solution). Such pharmaceutical agents can be dissolved within eutectic mixtures such as those described herein. For example, a delivery vehicle such as a patch, cream, lotion, gel, or the like may comprise a eutectic mixture, including pharmaceutical agents or other beneficial substances. The delivery vehicle may, for example, be applied to the skin of a subject, which may be used to deliver the pharmaceutical agent (or other beneficial substances) into the skin of the subject. For example, such a mixture may be orally taken, and thus can be delivered internally to the gastrointestinal tract. In some cases, the pharmaceutical agent may be dissolved within the eutectic mixture (i.e., “pre-dissolved”), and thus may be more readily bioavailable than if it were dissolved in water, and/or exhibit faster onset times (e.g., times before a biological effect due to the pharmaceutical agent can be observed). As another example, in some cases, the pharmaceutical agent or other beneficial substance may be dissolved within the eutectic mixture (i.e., “pre-dissolved”), and thus may be contained within the delivery vehicle for delivery into the skin of the subject.

Examples of pharmaceutical agents sensitive to water include, but are not limited to, loratadine, acetaminophen, or diphenhydramine.

In one set of embodiments, the pharmaceutical agent or beneficial substance may include small molecules (e.g., having a molecular weight of less than about 2,000 Da, less than about 1,500 Da, or less than about 1,000 Da), peptides (e.g., having less than about 10, less than about 15, less than about 20, or less than about 25 amino acids), proteins (typically larger than peptides), hormones, vitamins, nucleic acids, or the like.

Additional examples of suitable pharmaceutical agents include, but are not limited to, NSAIDs (nonsteroidal anti-inflammatory drugs) such as acetylsalicylsalicylic acid or aspirin, naproxen, celecoxib, rofecoxib, ketorolac, ibuprofen, diclofenac, acetaminophen, etc.; COX-1 and/or COX-2 inhibitors; pharmaceutical agents with narcotic action such as morphine, codeine, dihydrocodeine, propoxyphene, oxycodone, hydrocodone, or other similar narcotics; pharmaceutical agents for erectile or sexual dysfunction, including phosphodiesterase type 5 inhibitors, such as yohimbine, alprostadil, sildenafil, tadalafil, apomorphine, vardenafil, or the like; pharmaceutical agents for migraine such as dihydroergotamine (DHE) and its salts, ergotamine and its salts, sumatripan and its salts, rizatriptan and its salts, zolmitriptan and its salts, etc.; pharmaceutical agents for hair treatment such as finasteride, eflornithine, minoxidil, or the like; steroids such as fluticasone, cortisone, hydrocortisone, etc.; neurological agents such as gabapentin, pregabalin, etc.; migraine treatments such as triptans, DHE or dihydroergotamine, oxytocin, etc.; antifungals such as terbinafine or triazole antifungal drugs, etc.; pharmaceutical agents for allergy treatment, such as cetirizine, loratadine, diphenhydramine, etc.; pharmaceutical agents for treatment of skin diseases, such as methotrexate or acyclovir; pharmaceutical agents for wound healing, such as arginine, betahistine, histamine, etc.; or other pharmaceutical agents such as niacin, lidocaine, benzocaine, etc. Additional examples include muscle improving agents, for example, creatine or creatine precursors (e.g., creatine phosphate), arginine and/or other nitric oxide donors, and/or ATP precursors such as, inosine, adenosine, inosine, adenine, hypoxanthine, ribose, phosphate (e.g., monosodium phosphate), etc., and/or anabolic steroid agents, such as androstene, DHEA or dehydroepiandrosterone, androstenolone, androstenediol, androstenedione, or the like. Non-limiting examples of COX-2 inhibitors include celecoxib or rofecoxib. Non-limiting examples of COX-1/COX-2 inhibitors include ibuprofen, ketorolac, naproxen, diclofenac, aspirin, acetaminophen, etc. Another example is ephedra or its components, such as ephedrine and pseudoephedrine. Yet another example are chemotherapeutic agents or agents for treating cancer and/or viral infections, for example, but not limited to tamoxifen (e.g., for breast cancer treatment), cis-platin, carboplatin and related molecules, cyclophosphamide and related molecules, vinca alkaloids, epipodophyllotoxins including paclitaxel, aciclovir, or the like. For example, the cancer and/or viral infections may be skin cancer, breast cancer, penile cancer, testicular cancer, or other localized cancers, or viral infections, such as herpes.

In another set of embodiments, the pharmaceutical agent is a triptan and/or a salt of a triptan. Their action is attributed to their binding to serotonin 5-HT1B and 5-HT1D receptors in cranial blood vessels (causing their constriction) and subsequent inhibition of pro-inflammatory neuropeptide release. These drugs may act on serotonin receptors in nerve endings as well as the blood vessels, which may lead to a decrease in the release of several peptides, including CGRP and substance P. Triptans generally have a structure:

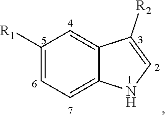

where R₁ may be a sulfonamide, a triazole (e.g., 1,2,3-triazole or 1,2,4-triazole), or a 2-oxazolidone; and R₂ may be a nitrogen-alkyl chain (e.g., —CH₂CH₂N(CH₃)₂), a dimethylpyrrolidine, or a 1-methyl-piperidine ring. A sulfonamide is generally a structure R^{a}SO₂NR^{b}R^{c}, where R^{a} may be an alkyl such as a C₁-C₅ alkyl (substituted or unsubstituted), for example, —CH₂—, —CH₂CH₂—, —CH₂CH₂CH₂—, etc., and R^{b} and R^{c} may each independently be —H or an alkyl such as a C₁-C₅ alkyl (substituted or unsubstituted), for example, —CH₃, —CH₂CH₃, etc., or an aryl group (substituted or unsubstituted) such as phenyl.

Non-limiting examples of triptans include sumatriptan (pKas of 6.16, 9.63, and 17.14), rizatriptan, naratriptan (pKa of 17.11), zolmitriptan (pKa of 17.15), eletriptan, almotriptan (pKa of 8.77), frovatriptan (pKa of 17.27), and avitriptan (pKas of 3.6 and 8.0). The structures of these compounds are respectively shown below:

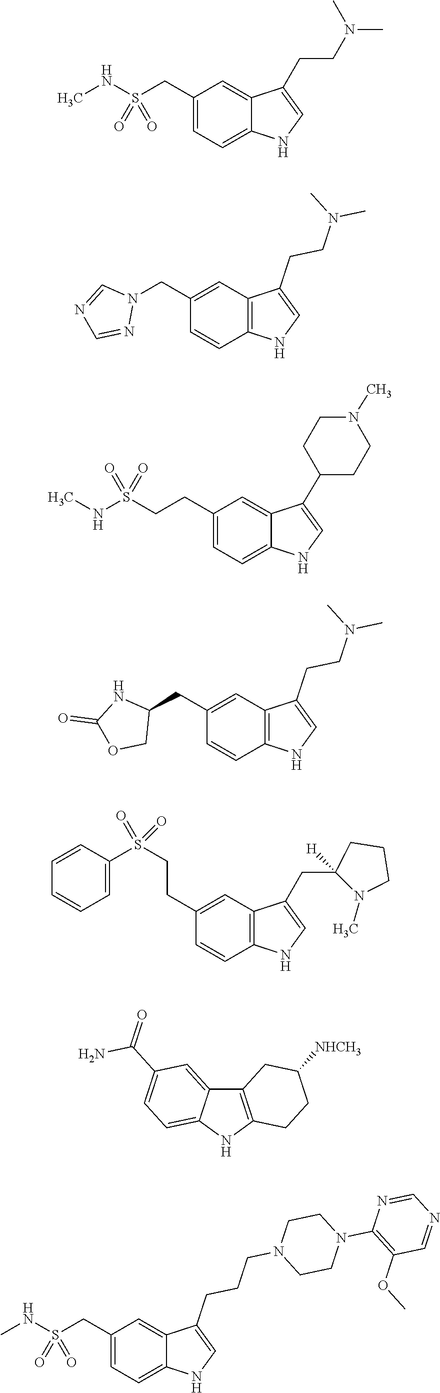

In some cases, the pharmaceutical agent may comprise a peptide. As one example, the peptide may be oxytocin (Cys-Tyr-Ile-Gln-Asn-Cys-Pro-Leu-Gly) or an oxytocin analog, such as 4-threonine-1-hydroxydeaminooxytocin, 4-serine, 8-isoleucine-oxytocin, 9-deamidooxytocin, 7-D-proline-oxytocin and its deamino analog, (2,4-diisoleucine)-oxytocin, deamino oxytocin analog, 1-deamino-1-monocarba-E12-Tyr(OMe)]-OT(dCOMOT), carbetocin (butyryl-Tyr(Me)-Ile-Gln-Asn-Cys-Pro-Leu-Gly-NH₂), 4-threonine, 7-glycine-oxytocin (TG-OT), (1-butanoic acid-2-(O-methyl-L-tyrosine)-1-carbaoxytocin, Ile-conopres sin, atosiban, oxypressin, deamino-6-carba-oxytoxin (dC60), desmopres sin, or 1-deamino-oxytocin.

Additional examples of peptides include, but are not limited to, vasopres sin, corticotropin releasing hormone (CRH), growth hormone releasing hormone (GHRH), luteinizing hormone releasing hormone (LHRH), somatostatin growth hormone release inhibiting hormone, thyrotropin releasing hormone (TRH), glial-derived neurotrophic factor (GDNF), brain-derived neurotrophic factor (BDNF), nerve growth factor (NGF), neurotrophin-3 (NT-3), pancreatic polypeptide, peptide tyrosine-tyrosine, glucogen-like peptide-1 (GLP-1), peptide histidine isoleucine (PHI), pituitary adenylate cyclase activating peptide (PACAP), brain natriuretic peptide, cholecystokinin (CCK), islet amyloid polypeptide (IAPP) or amylin, melanin concentrating hormone (MCH), melanocortins (ACTH, alpha-MSH and others), neuropeptide FF (F8Fa), neurotensin, parathyroid hormone related protein, Agouti gene-related protein (AGRP), cocaine and amphetamine regulated transcript (CART)/peptide, 5-HT-moduline, hypocretins/orexins, nociceptin/orphanin FQ, ocistatin, prolactin releasing peptide, secretoneurin, urocortin, or the like.

In one set of embodiments, the pharmaceutical agent is a triazole antifungal drug and/or a salt thereof. The “triazole” generally refers to a five-membered ring of two carbon atoms and three nitrogen atoms within the drug. Specific non-limiting examples of triazole antifungal drugs include fluconazole (pKas of 10.7-11.3, 2.8-3.0, and 2.0-2.6), isavuconazole, itraconazole (pKa of 3.7), voriconazole (pKa of 1.76), pramiconazole, or posaconazole (pKas of 3.6 and 4.6). The structures of these compounds are respectively shown below:

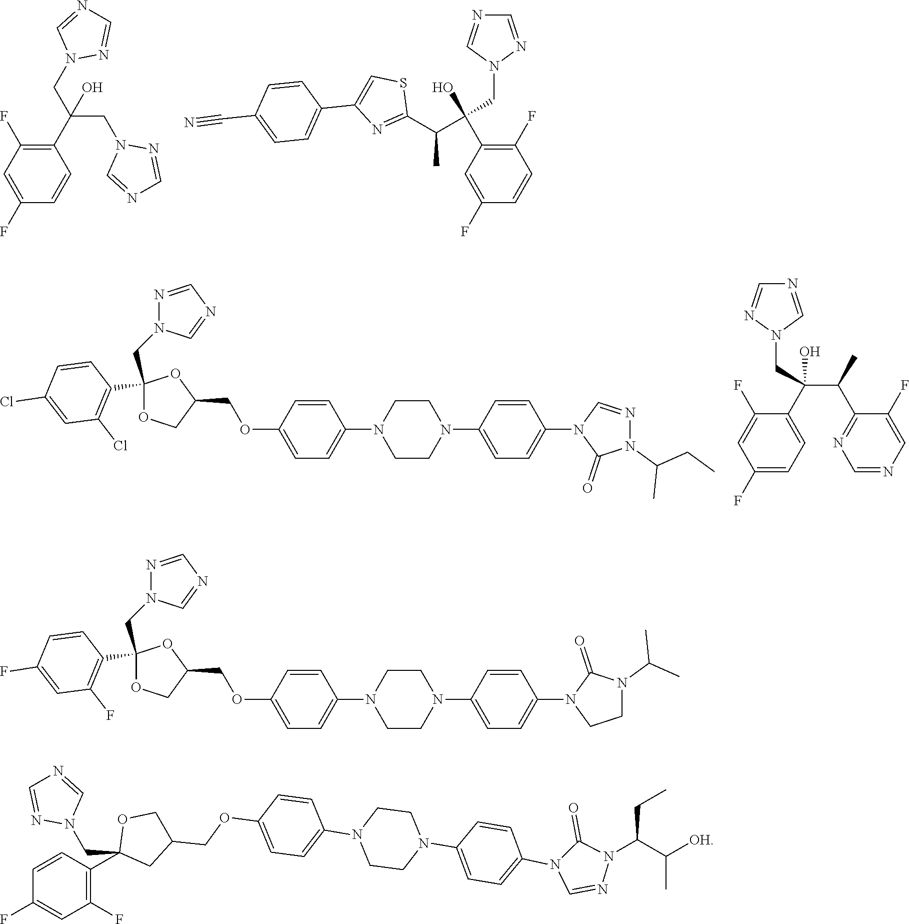

In one set of embodiments, the pharmaceutical agent is a terbinafine antifungal drug and/or a salt thereof. The structure of terbinafine is shown below:

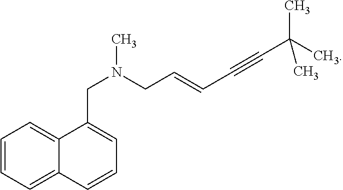 [SMILES: CN(C/C=C/C#CC(C)(C)C)Cc1cccc2ccccc12]

Still other examples of pharmaceutical agents include, but are not limited to, opioids, such as morphine, methadone, fentanyl, butorphanol, codeine, opium, oxycodone, loperimide, meperidine (Demerol), diphenoxylate, propoxyphene (Darvon), 4-methyl fentanyl, hydrocodone, morphine, diacetylmorphine, dihydrocodeine, hydromorphone (Dilaudid), levorphanol (Levo-Dromoran), dextromethorphan, oxymorphone (Numorphan), heroin, remifentanil, phenazocine, pentazocine, piminodine, anileridine, buprenorphine (Suboxone), sufentanil, carfentanil, alfentanil and the atypical opiates, tramadol and tapentadol; opioid and opioid-like peptides and their analogs, such as endorphins, enkephalins, dynorphins, dermorphins, dermenkephalins, morphiceptin, endomorphins and dalargin; NMDA-receptor antagonists, such as ketamine, amantadine, dextrometorphane, memantine and MK801; sodium channel blockers, such as local anesthetics and ergotamine; calcium channel blockers, such as verapamil and nifedipine; adrenergic antagonists, such as propranolol, metoprolol and yohimine; gabaergic agonists, such as GABA, baclofen, cis-4-aminocrotonic acid (CACA), trans-4-aminocrotonic acid (TACA), CGP 27 492 (3-aminopropyl phosphonous acid) and progabide; glycine agonists, such as glycine and D-cycloserine; cholinergic agonists, such as neostigmine and physiostigmine; adrenergic agonists, such as epinephrine, neosynephrine, clonidine and dexmedetomidine; anticonvulsants, such as gabapentin and barbiturates; Rho kinase inhibitors, such as fasudil, Y27632, H-1152 and derivatives thereof; PKC inhibitors, such as chelerythrine, Go 6983, GO 6976, N-myristoyl-Ser-Ile-Tyr-Arg-Arg-Gly-Ala-Arg-Arg-Trp-Arg-Lys-Leu, Rottlerin, KAI-9803 and KAI-1455; p38-MAP kinase inhibitors, such as SCIO-469, AMG548 and derivatives thereof; ATP receptor blockers, such as tetramethylpyrazine chelerythrine chloride, A-317491 and derivatives thereof; endothelin receptor blockers, such as BQ123, BMS182874 and derivatives thereof; pro-inflammatory cytokine, chemokine, interleukin and tumor necrosis factor blockers, such as anakinra, infliximab, etanercept and adalimumab; anti-inflammatory cytokines, such as interleukin-4, interleukin-10 and interleukin-13; tricyclic antidepressants, such as desiprimine and amitryptiline; serotonergic antagonists, such as fluoxetine, dolasetron and ondansetron; serotonergic agonists, such as buspirone and ergometrine; NSAIDs and COXIBs, such as diclofenac, ibuprofen, ketorolac, salicylate, rofecoxib, celecoxib, parecoxib, valdecoxib and naproxen; acetaminophen; analgesic peptides, such as calcitonin, octreotide, somatostatin, vasopressin, galanin, the C-fragment of lipotropin and Ac-rfwink-NH₂; toxins, such as botulinum toxin, variants and derivatives thereof, cone snail toxins, such as omega-conotoxin GV1A, omega-conotoxin MVIIA, saxitoxin and tetrodotoxin; TRP channel agonists and antagonists, such as capsaicin, capsazepine, resiniferotoxin, SB-705498, A-425619, AMG 517, SC0030 and derivatives thereof; cannabanoids, such as THC, CT-3, levonantradol, dexanabinol, WIN-55,212-2, AM 1241, dronabinol, nabilone, cannabis medicinal extract (CME) and derivatives thereof; antagonists of pro-nociceptive peptide neurotransmitter receptors CGRP1 and CGRP2, including non-peptide antagonists such as BIBN4096 and derivatives thereof and peptide antagonists such as CGRP 8-37 and CGRP 28-3; antagonists of pro-nociceptive peptide neurotransmitter receptor NK1, including non-peptide antagonists such as SR140333, CP96346, L-760735; RP 67580, WIN 51708; MK869, and derivatives thereof and peptide antagonists such as N-acetyl tryptophan, D-Pro9-[Spiro-y-lactam]-Leu 10,Trp 11-Physalaemin(1-11), Tyr-D-Phe-Phe-D-His-Leu-Met-NH₂ (Sendide) and spantide II; antagonists of pro-nociceptive peptide neurotransmitter receptor NK2, including non-peptide antagonists such as SR 48968 and derivatives thereof and peptide antagonists such as PhCO-Ala-Ala-D-Trp-Phe-D-Pro-Pro-Nle-NH₂ (GR98400), [Tyr5,D-Trp6,8,9,Lys10]-NKA (4-10) (MEN10376) and derivatives thereof; antagonists of pro-nociceptive peptide neurotransmitter receptor Y1-5, including non-peptide antagonist benextramine and peptide antagonists (Ile-Glue-Pro-Dpr-Tyr-Arg-Leu-Arg-Tyr-NH₂)₂, cyclic (2,4′),(2,4′)-diamide (1229U91 or GW1229), PYX-2, D-Tyr (27,36), D-Thr (32)] NPY (27-36)(D-NPY(27-36), 3-(5,6,7,8-tetrahydro-9-isopropyl-carbazol-3-yl)-1-methyl-1-(2-pyridin-4-yl-ethyl)-urea hydrochloride (FMS586 and derivatives thereof); antagonists of pro-nociceptive peptide neurotransmitter receptors VPAC2, VPAC1 and PAC1, including peptide antagonists VIP(6-28), Ac His(1) [D-Phe(2), K(15), R(16), L(27)] VIP (3-7)/GRF (8-27); antagonists of pro-nociceptive neurotransmitter receptors Gall-3 and GalR1-3, including non-peptide antagonists SNAP 37889, SNAP 398299, galnon and derivatives thereof.

The above compounds may be present within the composition in any suitable amounts. For example, one or more of the above compositions may be present at at least about 0.1%, at least about 0.2%, at least about 0.3%, at least about 0.5%, at least about 1%, at least about 2%, at least about 3%, at least about 5%, at least about 10%, etc. In some cases, the compositions may be present at no more than about 10%, no more than about 5%, no more than about 3%, no more than about 2%, no more than about 1%, no more than about 0.5%, no more than about 0.3%, no more than about 0.2%, or no more than about 0.1%. Combinations of any of these percentages are also possible. The actual concentration for a particular application can be determined by those of ordinary skill in the art using no more than routine experimentation, for example, by measuring the amount of transport of a compound as a function of concentration in vitro across cadaver skin or suitable animal models, skin grafts, synthetic model membranes, human models, or the like.

The composition may comprise a hostile biophysical environment to the substance in some embodiments. In a hostile biophysical environment, the environment surrounding the substance may be such that the pharmaceutical agent is in a chemically and/or energetically unfavorable environment, relative to the skin (e.g., the chemical potential and/or the free energy of the pharmaceutical agent within the hostile biophysical environment is significantly greater than the chemical potential and/or the free energy of the substance within the skin, thus energetically favoring transport into the skin), especially the stratum corneum.

Techniques for hostile biophysical environments are discussed in detail herein. For example, certain embodiments of the invention are generally directed to compositions for topical delivery to the skin of a subject comprising a nitric oxide donor and a hostile biophysical environment.

A hostile biophysical environment of the invention can comprise, in one set of embodiments, a high ionic strength environment. For example, the composition may have an ionic strength of at least about 0.25 M, at least about 0.5 M, at least about 1 M, at least about 2 M, at least about 3 M, at least about 4 M, at least about 5 M, at least about 7 M, at least about 10 M, at least about 12 M, at least about 15 M, at least about 20 M, at least about 25 M, etc. In some cases, the ionic strength of the composition may be no more than about 25 M, no more than about 20 M, no more than about 15 M, no more than about 12 M, no more than about 10 M, no more than about 7 M, no more than about 5 M, no more than about 4 M, no more than about 3 M, no more than about 2 M, no more than about 1 M, no more than about 0.5 M, no more than about 0.25 M, etc. Combinations of any of these ionic strengths are also possible. For example, the ionic strength may be between about 0.25 M and about 15 M, between about 5 M and about 15 M, between about 10 M and about 15 M, etc. In some embodiments, the ionic strength is any amount greater than two times the physiological ionic strength of blood.

In some embodiments, the high ionic strength environment may be caused by the presence or one or more salts within the composition. For instance, the ionic strength of a composition can be readily controlled in certain embodiments by controlling the amounts or concentrations of one or more of the salts present in the composition. Examples of ionic salts that may be used include, but are not limited to, one or more of sodium chloride, magnesium chloride, potassium chloride, calcium chloride, choline chloride, lithium chloride, sodium bromide, magnesium bromide, potassium bromide, calcium bromide, choline bromide, lithium bromide, sodium iodide, magnesium iodide, potassium iodide, calcium iodide, choline iodide, lithium iodide, sodium citrate, magnesium citrate, potassium citrate, calcium citrate, choline citrate, lithium citrate, etc. Combinations of these and/or other salts may also be used in certain embodiments. In addition, other highly charged molecules such as polylysine, polyglutamine, polyaspartate, etc., or copolymers of such highly charged amino acids may also be used in certain embodiments.

In some cases, one or more salts may each independently be present at a concentration of at least about 0.25 M, at least about 0.5 M, at least about 1 M, at least about 2 M, at least about 3 M, at least about 4 M, at least about 5 M, at least about 7 M, at least about 10 M, at least about 12 M, at least about 15 M, at least about 20 M, at least about 25 M, etc. In some cases, the concentration may be no more than about 25 M, no more than about 20 M, no more than about 15 M, no more than about 12 M, no more than about 10 M, no more than about 7 M, no more than about 5 M, no more than about 4 M, no more than about 3 M, no more than about 2 M, no more than about 1 M, no more than about 0.5 M, no more than about 0.25 M, etc. Combinations of any of these concentrations are also possible. For example, the ionic strength may be between about 0.25 M and about 15 M, between about 5 M and about 15 M, between about 10 M and about 15 M, etc. In addition, it should be noted that if more than one salt is present, each contributing to the overall ionic strength, then the concentrations of each may be independent of each other. In some cases, the concentrations of each may be relatively low although the sum total contribution of each to ionic strength may be used to produce a high ionic strength environment.

Thus, according to some embodiments of the invention, compositions comprising a relatively high salt composition (e.g., high chloride content) are effective for topical delivery of certain compounds. In some embodiments, salt-enhanced delivery is particularly effective. In some cases, one or more salts may each be present within composition at at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (by weight). In some embodiments, one or more salts may each independently be present at no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1%. Combinations of any of these ranges are also possible in certain embodiments. For example, a salt such as sodium chloride may be present at between 5 wt % and 15 wt % in the composition. In addition, in some cases, the total amount of salt present within the composition may be within any of these ranges. Thus, for example, one or more salts (e.g., sodium chloride and/or potassium chloride) may be present within the composition such that the total amount of salt is between 5 wt % and 15 wt %.

In some cases, the pH of the composition may be optimized to ionize the compound being delivered (e.g., at least about 80%, at least about 90%, at least about 95%, or about 99% or more) is ionized. It should be appreciated that depending on the pKa of the compound and the pH of the composition, the ionized form may be anionic or cationic (e.g., due to protonation). In some embodiments, a compound may contain several ionizable groups each having a different pKa. In some embodiments, it is sufficient for at least 1, 2, or 3 of the groups to be ionized for the salt-enhanced delivery to be effective. In certain embodiments, an ionizable group is sufficiently ionized if the pH of the composition is at least 1 pH unit, or at least 2 pH units (e.g., 1, 1-2, 2-3, or more pH units) below the pKa of the group and it is cationic (due to protonation) below its pKa. Similarly, in some embodiments, an ionizable group is sufficiently ionized if the pH of the composition is at least 1 pH unit, or at least 2 pH units (e.g., 1, 1-2, 2-3, or more pH units) above the pKa of the group and it is anionic (due to deprotonation) above its pKa. In some embodiments, the presence of magnesium chloride, for example at 0.1-5% by weight, can help stabilize compositions containing compounds with relatively high pKas (e.g., above 8.0, above 9.0, above 10.0 or higher). In some embodiments, the pH of a composition may be maintained using a buffer. However, the pH of some compositions of the invention may be stable without a buffer. In some embodiments, a desired pH can be established by titrating the mixture with an acid (e.g., HCl) or a base (e.g., NaOH). The pH of the resulting composition (e.g., when formulated as an emulsion as described herein) can be stable (e.g., sufficiently for the composition to be effective for transdermal delivery) for extended periods of time (e.g., weeks, months, or 1 or more years).

In other embodiments, other methods may be used to produce a hostile biophysical environment, e.g., in addition to or instead of a high ionic strength environment. For instance, in one set of embodiments, a hostile biophysical environment may be produced using a high concentration of osmotic agents such as ureas, sugars, or carbohydrates, a high pH environment (e.g., greater than about 7, greater than about 8, greater than about 9, greater than about 10, greater than about 11, greater than about 12, or greater than about 13), a low pH environment (less than about 5, less than about 4, less than about 3 or less than about 2), highly hydrophobic components, or highly hydrophilic components or other substances that cause an increase in the chemical potential and/or free energy of the pharmaceutical agent, or any combination of two or more of these and/or other compounds. A hydrophobic component may, in some embodiments, have an octanol-water partition coefficient of at least about 100, at least about 1000, at least about 10⁴, at least about 10⁵, or more in some cases. Similarly, a hydrophilic component may have an octanol-water partition coefficient of less than about 0.01, less than about 10⁻³, less than about 10⁴, or less than about 10⁻⁵ in some cases. In some cases, high or low pH environments (e.g., by adding pharmaceutically acceptable acids or bases, for example, such that the pH is between about 3 and about 7, between about 3 and about 6, between about 3 and about 5, between about 4 and 8, between about 5 and about 8, between about 5 and 8.5, between about 7 and about 11, between about 8 and about 11, between about 9 and about 11, etc.); or highly hydrophobic environments (e.g., by decreasing water content and increasing lipid, oil and/or wax content of the environment) may be used to create a biophysical hostile environment.

In some cases, the composition defines the biophysical hostile environment. In certain cases, a substance may be packaged in such a way that it is carried into tissue and/or its charge is neutralized by derivitization and/or by forming a neutral salt. Non-limiting examples of delivery vehicles which would be carried into tissue includes liposomes or emulsions of collagen, collagen peptides or other components of skin or basement membrane. Non-limiting examples of neutralization of charge include delivery of the substance in the form or an ester or salt which is electronically neutral. In some embodiments, the hostile biophysical environment may include any two or more of these conditions. For instance, the hostile biophysical environment may include high ionic strength and a high pH or a low pH, a highly hydrophobic environment and a high pH or a low pH, a highly hydrophobic environment that includes liposomes, or the like.

A hostile biophysical environment may also be created in some embodiments by placing a substance that is relatively highly charged into a hydrophobic, oily environment such as in an oil-based cream or lotion containing little or no water. Absorption may further be aided in some cases by combining the use of hostile biophysical environments with the use of penetrating agents, as further described herein.

In some embodiments, the composition may include one or more penetrating agents, e.g., that are able to increase transport across the skin, relative to transport in the absence of the penetrating agent. For instance, transport of the nitric oxide donor, and/or other substances such as those described herein, may be enhanced using one or more penetration agents. Non-limiting examples of penetrating agents include, but are not limited to, cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); fatty acid esters (e.g., n-butyrate); organic acids (e.g., citric acid); polyols (e.g., ethylene glycol, propylene glycol, glycerol); sulfoxides (e.g., dimethylsulfoxide); terpenes (e.g., cyclohexene); ureas; sugars; carbohydrates or other agents (e.g., azone or laurocapram). In certain embodiments, the penetrating agent includes a salt, e.g., as described herein. Additional examples of penetrating agents include certain molecules containing heterocyclic rings to which are attached hydrocarbon chains.

The pharmaceutical agents or beneficial substances may be present in any amount or concentration. For example, a pharmaceutical agent or or a beneficial substance may be present within a liquid formulation at at least 0.01 mol %, at least 0.02 mol %, at least 0.03 mol %, at least 0.05 mol %, at least 0.1 mol %, at least 0.2 mol %, at least 0.3 mol %, at least 0.5 mol %, at least 1 mol %, at least 2 mol %, at least 3 mol %, at least 5 mol %, at least 10 mol %, at least 15 mol %, at least 20 mol %, at least 25 mol %, etc. In addition, in certain embodiments, the pharmaceutical agent or beneficial substance may be present within a liquid formulation at no more than 25 mol %, no more than 20 mol %, no more than 15 mol %, no more than 10 mol %, no more than 5 mol %, no more than 3 mol %, no more than 2 mol %, no more than 1 mol %, no more than 0.5 mol %, no more than 0.3 mol %, no more than 0.2 mol %, no more than 0.1 mol %, no more than 0.05 mol %, no more than 0.03 mol %, no more than 0.02 mol %, no more than 0.01 mol %, etc. Combinations of any of these ranges are also possible in various embodiments; for example, one (or more) of the pharmaceutical agent and/or beneficial substances may be present at between 1 mol % and 20 mol %, between 5 mol % and 20 mol %, between 5 mol % and 10 mol %, between 10 mol % and 25 mol %, between 0.5 mol % and 2 mol %, etc., of the liquid formulation. As a non-limiting example, aspirin may be present at between 1 mol % and 5 mol %, between 1 mol % and 10 mol %, etc.

In some embodiments, the liquid formulation may comprise or consist essentially of a eutectic mixture and one or more pharmaceutical agents and/or beneficial substances. Water may or may not be present; if water is present, in certain embodiments, the amount of water that is present may be very low, e.g., less than 2 mol % or 1 mol %. Thus, the liquid formulation may be substantially anhydrous in some embodiments. In certain embodiments, the percentages of eutectic components, pharmaceutical agents and/or beneficial substances, and water (if present) may sum to at least 50 mol %, at least 60 mol %, at least 70 mol %, at least 75 mol %, at least 80 mol %, at least 85 mol %, at least 90 mol %, at least 95 mol %, at least 97 mol %, or at least 99 mol %, or 100 mol % of the liquid formulation.

As a non-limiting example, in one set of embodiments, a formulation may comprise urea and choline as eutectic components, and aspirin as a pharmaceutical agent (alone, or with other pharmaceutical agents such as caffeine and/or acetaminophen, and/or other beneficial substances). For instance, the formulation may comprise 5 mol % to 20 mol % of pharmaceutical agents and/or beneficial substances, 5 mol % to 95 mol % choline chloride, and 5 mol % to 95 mol % urea. Water may be present, or the liquid formulation may be substantially anhydrous. In some cases, water is present at less than 1 mol %. The percentages of the pharmaceutical agents, choline chloride, urea, and water sum to at least 80 mol %, at least 90 mol %, or at least 100 mol % of the formulation.

Such formulations may be prepared by any suitable technique. For example, in certain embodiments, a eutectic may be prepared by combining the two or more components of the eutectic together (e.g., as a mixture of solids), and supplying heat. Mixing the components to produce the eutectic may be endothermic in embodiments, such that some degree of heat may be needed to facilitate mixing and formation of the eutectic. For instance, the mixture may be heated to at least 30° C., at least 40° C., at least 50° C., at least 60° C., at least 70° C., at least 80° C., at least 90° C., at least 100° C., etc. In some cases, ambient temperatures may be sufficient to cause the eutectic to form. In certain embodiments, it may take at least 1 hour, at least 2 hours, at least 6 hours, at least 12 hours, at least 24 hours, etc. for the eutectic to form.

In some aspects, liquid formulations such as those described above may be administered to a subject, such as a human subject. In one set of embodiments, the formulation may be administered as a liquid to a subject. Thus, for example, the formulation may be taken orally by a subject (e.g., drank by the subject). In some cases, for instance, the formulation may be prepared as a beverage, or contained within a bottle, vial, can, or the like, for oral administration. In some embodiments, as discussed herein, components that are sensitive to water and/or are not easily soluble in water may be administered, e.g., in liquid form, using eutectic mixtures such as those described herein. This may facilitate the delivery of such components, e.g., orally.

In some cases, however, the formulation is used to form other delivery vehicles, for example, patches, creams, lotions, gels, and the like. For instance, a liquid may be mixed with a suitable adhesive compound and used to make a patch. As further examples, a liquid formulation may be formed within creams, lotions, gels, and the like. In some cases, as discussed herein, such liquid formulations may be substantially anhydrous. For example, such formulations may have water contents of less than 5 mol %, less than 2 mol %, or less than 1 mol %, or other water concentrations such as those discussed herein. For example, as discussed herein, components that are sensitive to water and/or are not easily soluble in water may be present in such delivery vehicles.

For instance, in one embodiment, a eutectic mixture may be mixed with a suitable adhesive compound and used to make a patch, or a bandage. As further examples, a formulation such as described herein may be formed within creams, lotions, gels, and the like. In some cases, as discussed herein, such formulations may be substantially anhydrous. For example, such formulations may have water contents of less than 5 wt %, less than 2 wt %, or less than 1 wt %, or other water concentrations such as those discussed herein. In some cases, the transdermal patch or bandage may comprise a backing layer and an adhesive. The adhesive may in some cases contain a pharmaceutical agent (or other beneficial substance), or there may be a separate layer or portion of the patch or bandage that contains the pharmaceutical agent or other beneficial substance. Those of ordinary skill in the art will be familiar with transdermal patches, bandages, and the like.

For example, non-limiting examples of adhesives include, but are not limited to polyacrylate polymers, rubber-based adhesives and polysiloxane adhesives, natural or synthetic polyisoprene, polybutylene, polyisobutylene, styrene-butadiene polymers, styrene based polymers, styrene block copolymers, butadiene based polymers, styrene/butadiene polymers, styrene-isoprene-styrene block copolymers, hydrocarbon polymers, such as butyl rubber, halogen-containing polymers such as, for example, polyacrylonitrile, polytetrafluoroethylene, polyvinylchloride, polyvinylidene chloride, and polychlorodieneas polyisobutylenes, polybutylenes, ethylene/vinyl acetate and vinyl acetate based adhesives, styrene/butadiene adhesives, etc.

In one set of embodiments, the eutectic mixture is used within an emulsion, e.g., to form a cream, lotion, or the like. As known by those of ordinary skill in the art, an emulsion typically includes a first phase (e.g., a discontinuous phase) contained within a second fluid phase (e.g., a continuous phase). The substance may be present in either or both phases. In addition, other materials such as those described herein may be present in the same phase as the substance. In some embodiments, the emulsion may take the form of a cream or a lotion. For example, a substance may be contained within a hydrophobic, oily environment such as in an oil-based cream or lotion containing little or no water.

For example, a cream may include materials such as oils, triglycerides, stearates, fatty acids, fatty alcohols, squalenes, polysorbates, or the like. In some cases, such materials are hydrophobic, which can be emulsified with water or other aqueous phases, e.g., to produce an emulsion. In one embodiment, for example, the cream may include a saturated squalene. Examples of stearates include, but are not limited to, glyceryl stearate, propylene glycol stearate, steryl stearate, sorbitan stearate, sodium stearate, calcium stearate, magnesium steratae, glycol sterate, and the like. Non-limiting examples of oils include mineral oil, wheat germ oil, palm oil, nut oil, linseed oil, etc. Other materials may also be present within the composition, for example, buffers, preservatives, surfactants, etc.

In one set of embodiments, as a non-limiting example, a cream may include one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A and D, triethanolamine, methylparaben, aloe vera extract, imidazolidinyl urea, propylparaben, PND, and/or BHA. As another example, a cream may include one or more of water, sodium chloride, potassium chloride, L-arginine HCl, mineral oil, caprylic/capric triglycerides, phenoxyethanol, glycerol stearate, PEG 75 stearate, cetyl alcohol, methylparaben, and propylparaben.

In another aspect, a composition as described herein may be administered to a subject, either by itself and/or in conjunction with co-factors, other therapeutics, or the like. In some cases, the composition includes a pharmaceutically acceptable eutectic mixture, e.g., as described herein. For example, a liquid formulation may be administered alone, or in conjunction with other formulations. As another example, in certain embodiments, the composition may be applied in the form of a patch, cream, lotion, or gel, or the like, such as described herein. When administered, the compositions can be applied in a therapeutically effective, pharmaceutically acceptable amount as a pharmaceutically acceptable formulation, for example, a pharmaceutically acceptable carrier such as those described below. The term “effective amount” of a composition, such as the complexes described herein, refers to the amount necessary or sufficient to realize a desired biologic effect. For example, an effective amount of aspirin to treat pain may be an amount sufficient to alleviate or reduce the sensation of pain. In some cases, at least some of the pain may be subjective. Combined with the teachings provided herein, by choosing among the various active compositions and weighing factors such as potency, relative bioavailability, patient body weight, severity of adverse side effects and mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular compositions being administered the size of the subject, or the severity of the disease or condition. One of ordinary skill in the art can empirically determine the effective amount of the compositions without necessitating undue experimentation.

The terms “treat,” “treated,” “treating,” and the like, when used herein, refer to administration of the compositions to a subject which may increase the resistance of the subject to development or further development of the disease or condition, to administration of the composition after the subject has developed the disease or condition in order to eliminate or at least control development of the disease or condition, and/or slow the progression of or to reduce the severity of symptoms caused by the disease or condition. When administered to a subject, effective amounts may depend on the particular disease or condition being treated and the desired outcome. A therapeutically effective dose may be determined by those of ordinary skill in the art, for instance, employing factors such as those further described below and using no more than routine experimentation.

For use in therapy, an effective amount of the compositions can be administered to a subject by any mode that delivers the composition to the subject, e.g., oral, topical, transdermal, or the like. Administering the pharmaceutical composition may be accomplished by any method. Example routes of administration include but are not limited to oral, parenteral, intramuscular, intranasal, sublingual, intratracheal, inhalation, ocular, vaginal, intravenously, percutaneously, and rectal.

As another example, a liquid, gel, or the like, such as is described herein, may be contained within a capsule that can be orally administered to a subject. For instance, in one set of embodiments, a deep eutectic mixture may be contained within a capsule, such as a hard capsule or a soft capsule. The capsule may comprise, for instance, gelatin, hypromellose, pullulan, carrageenans, starch, cellulose, or other materials known to those of ordinary skill in the art.

Administering the pharmaceutical composition may be accomplished by any method. In administering the compositions to a subject, dosing amounts, dosing schedules, routes of administration, and the like may be selected so as to affect known activities of these compositions. Dosages may be estimated based on the results of experimental models, optionally in combination with the results of assays of compositions described herein. Dosage may be adjusted appropriately to achieve desired drug levels, local or systemic, depending upon the mode of administration. The doses may be given in one or several administrations per day. Multiple doses per day are also contemplated in some cases to achieve appropriate systemic levels of the compositions within the subject.

The dose of the compositions to the subject may be such that a therapeutically effective amount of the compositions reaches the subject. The dosage may be given in some cases at the maximum amount while avoiding or minimizing any potentially detrimental side effects within the subject. The dosage of the compositions actually administered may be dependent upon factors such as the final desired concentration, the method of administration to the subject, the efficacy of the composition, the longevity of the composition within the subject, the timing of administration, the effect of concurrent treatments, etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition. Variations in dosing may occur between different individuals or even within the same individual on different days. In some cases, a maximum dose be used, that is, the highest safe dose according to sound medical judgment. In some cases, the dosage form is such that it does not substantially deleteriously affect the subject.

Subject doses of the compounds described herein for delivery may range from about 0.1 microgram to 10 mg per administration, which depending on the application could be given daily, weekly, or monthly and any other amount of time therebetween. In some cases, doses range from about 10 microgram to 5 mg per administration, e.g., from about 100 microgram to 1 mg, with 2 to 4 administrations being spaced days or weeks apart. In some embodiments, doses range from 1 microgram to 10 mg per administration, and most typically 10 microgram to 1 mg, with daily or weekly administrations. Other suitable dosings have been described in detail herein.

The compositions may be administered in multiple doses over extended period of time. For any compound described herein the therapeutically effective amount can be initially determined from animal models. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The treatments disclosed herein may be given to any subject, for example, a human, or a non-human animal, such as a dog, a cat, a horse, a rabbit, a cow, a pig, a sheep, a goat, a rat (e.g., Rattus Norvegicus), a mouse (e.g., Mus musculus), a guinea pig, a non-human primate (e.g., a monkey, a chimpanzee, a baboon, an ape, a gorilla, etc.), or the like.

Administration of a composition of the invention may be accomplished by any medically acceptable method. The particular mode selected may depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the compositions within the subject without causing clinically unacceptable adverse effects.

In some aspects, a composition as discussed herein is administered to a subject. Such administration may be systemic or localized, e.g., directed to a specific location of the body of a subject. The composition may be applied in any suitable form, e.g., as discussed herein. For example, the composition may be applied using a delivery vehicle such as a cream, gel, liquid, lotion, spray, aerosol, or transdermal patch. In one set of embodiments, a composition may be applied or impregnated in a bandage or a patch applied to the skin of a subject. In some embodiments, a patch has a skin contacting portion made of any suitable material that is covered or impregnated with a cream or emulsion described herein, wherein the skin contacting portion may be supported by a backing, one or both of which may have an adhesive segment or other configuration for attaching to the skin surface of a subject.

Such delivery vehicles may be applied to the skin of a subject, such as a human subject. Examples of delivery vehicles are discussed herein. The delivery vehicle may promote transfer into the skin of an effective concentration of the pharmaceutical agent or beneficial substance, directly or indirectly. For instance, the delivery vehicle may include one or more penetrating agents, as further described herein. Those of ordinary skill in the art will know of systems and techniques for incorporating a nitric oxide donor and/or a pharmaceutical agent within delivery vehicles such as a cream, gel, lotion, spray, aerosol, or transdermal patch.

Any medically acceptable method may be used to administer the compositions to the subject. The administration may be localized (i.e., to a particular region, physiological system, tissue, organ, or cell type) or systemic, depending on the condition to be treated. For example, the compositions may be administered orally, vaginally, rectally, buccally, pulmonary, topically, nasally, transdermally, through parenteral injection or implantation, via surgical administration, or any other method of administration. In some cases, more than one method of administration may be used, e.g., if two or more compositions are to be administered.

Examples of parenteral modalities that can be used include intravenous, intradermal, subcutaneous, intracavity, intramuscular, intraperitoneal, epidural, or intrathecal. Examples of implantation modalities include any implantable or injectable drug delivery system. Oral administration may be used in some embodiments because of the convenience to the subject as well as the dosing schedule. Compositions suitable for oral administration may be presented as discrete units such as hard or soft capsules, pills, cachettes, tablets, troches, or lozenge. Other oral compositions suitable for use include solutions or suspensions in aqueous or non-aqueous liquids such as a syrup, an elixir, or an emulsion. In another set of embodiments, a composition may be used to fortify a food or a beverage.

In one set of embodiments, the compositions are administered by inhalation. For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of a composition and a suitable powder base such as lactose or starch.

Also contemplated herein is pulmonary delivery. The compositions may be delivered to the lungs of the subject while inhaling and traverses across the lung epithelial lining to the blood stream.

Also contemplated for use are a wide range of mechanical devices designed for pulmonary delivery of therapeutic products, including but not limited to nebulizers, metered dose inhalers, powder inhalers, etc. The devices may require the use of formulations suitable for the dispensing of the compositions described herein. Typically, a formulation may involve the use of an appropriate propellant material, in addition to the usual diluents, adjuvants, carriers, etc. useful in therapy. Also, the use of liposomes, microcapsules or microspheres, inclusion complexes, or other types of carriers are contemplated for certain embodiments.

Formulations suitable for use with a nebulizer, including jet or ultrasonic, may comprise a composition as described herein. The nebulizer formulation may also contain a surfactant in certain embodiments to reduce or prevent surface induced aggregation of the compositions, e.g., as caused by atomization of the formulation in forming the aerosol.

Formulations for use with a metered dose inhaler device may generally comprise a powder containing a composition as described herein, which may be suspended in a propellant, optionally with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device may comprise a composition as described herein. In some cases, the device may also include a bulking agent, such as lactose, sorbitol, sucrose, or mannitol in amounts which facilitate dispersal from the device.

Nasal delivery of a composition as described herein is also contemplated. Nasal delivery allows the passage of a composition to the blood stream directly after administering the therapeutic product to the nose, without the necessity for deposition of the product in the lung. Formulations for nasal delivery include those with dextran or cyclodextran.

For nasal administration, a useful device for certain embodiments is a small, hard bottle to which a metered dose sprayer is attached. In one embodiment, the metered dose is delivered by drawing a composition as described herein into a chamber of defined volume, which chamber has an aperture dimensioned to aerosolize and aerosol formulation by forming a spray when a liquid in the chamber is compressed. The chamber may be compressed to administer the composition. In a specific embodiment, the chamber is a piston arrangement.

In another embodiment, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. In some cases, the nasal inhaler may provide a metered amount of the aerosol formulation, for administration of a measured dose of the composition.

For oral administration, a composition as described herein can be formulated readily by combining active composition with pharmaceutically acceptable carriers well known in the art. Such carriers enable the composition to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as crosslinked polyvinylpyrrolidone, agar, or alginic acid, or a salt thereof such as sodium alginate. Optionally, the oral formulations may also be formulated in saline or buffers, e.g., EDTA for neutralizing internal acid conditions or may be administered without any carriers.

Also specifically contemplated are oral dosage forms of the above compositions. The compositions may be chemically modified, in some embodiments, so that oral delivery of the derivative is efficacious. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Other polymers that could be used are poly-1,3-dioxolane and poly-1,3,6-tioxocane.

The location of delivery may be the stomach, the small intestine (the duodenum, the jejunum, or the ileum), or the large intestine. In some cases, formulations may be used which may not dissolve in the stomach, yet can release the material in the duodenum or elsewhere in the intestine. In some cases, the release may avoid the deleterious effects of the stomach environment, either by protection of the compositions of the present invention, and/or by release of the biologically active material beyond the stomach environment, such as in the intestine.

Colorants and/or flavoring agents may be included. For example, compositions as described herein may be formulated (such as by liposome or microsphere encapsulation) and then further contained within an edible product, such as a refrigerated beverage containing colorants and flavoring agents.

The volume may be diluted on increased with an inert material. Suitable diluents include carbohydrates, especially mannitol, a lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Pharmaceutical preparations which can be used orally include push fit capsules made of gelatin, as well as soft, sealed capsules made of gelatin and a plasticizer, such as glycerol or sorbitol. The push fit capsules can contain the compositions as described herein in admixture with filler such as lactose, binders such as starches, and/or lubricants such as talc or magnesium stearate and, optionally, stabilizers. In soft capsules, the active compositions may be dissolved or suspended in suitable liquids, such as fatty oils, liquid paraffin, or liquid polyethylene glycols. In addition, stabilizers may be added in some cases. Microspheres formulated for oral administration may also be used in some embodiments. The formulations for oral administration may be in dosages suitable for such administration.

The pharmaceutical compositions also may comprise suitable carriers or excipients in some embodiments. Examples of such carriers or excipients include, but are not limited, to calcium carbonate, calcium phosphate, various sugars, starches, cellulose derivatives, gelatin, and polymers such as polyethylene glycols.

Suitable liquid pharmaceutical preparation forms include, for example, liquid formulations, solutions, microencapsulated, encochleated, coated onto microscopic gold particles, contained in liposomes, nebulized, aerosols, or the like. The pharmaceutical compositions also may include tablets, coated tablets, (micro)capsules, suppositories, syrups, emulsions, suspensions, creams, drops or preparations, in which preparation excipients and additives and/or auxiliaries such as disintegrants, binders, coating agents, swelling agents, lubricants, flavorings, sweeteners and/or solubilizers can be added in some embodiments.

In certain embodiments of the invention, the administration of a composition as described herein may be designed so as to result in sequential exposures to the composition over a certain time period, for example, hours, days, weeks, months, or years. This may be accomplished, for example, by repeated administrations of the composition by one of the methods described herein, or by a sustained or controlled release delivery system in which a composition is delivered over a prolonged period without repeated administrations. Administration of a composition using such a delivery system may be, for example, by oral dosage forms, or other methods such as those described herein. Maintaining a substantially constant concentration of a composition may be desired in some cases.

In certain embodiments of the invention, a composition can be combined with a suitable pharmaceutically acceptable carrier, for example, as incorporated into a liposome, incorporated into a polymer release system, or suspended in a liquid, e.g., in a dissolved form or a colloidal form, or other methods such as those described herein. In general, pharmaceutically acceptable carriers suitable for use are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within a composition before use. The carrier may include one or more compatible solid or liquid fillers, diluents or encapsulating substances which are suitable for administration to a human or other vertebrate animal. The term “carrier” denotes an organic or inorganic ingredient, which may be natural or synthetic, with which one or more active compounds of the invention are combined to facilitate application. The carrier may be co-mingled or otherwise mixed with one or more compositions as described herein, and/or with each other, in a manner such that there is no interaction which would substantially impair the desired pharmaceutical efficacy. The carrier may be either soluble or insoluble, depending on the application. Examples of well-known carriers include, but are not limited to, glass, polystyrene, polypropylene, polyethylene, dextran, nylon, amylase, natural and modified cellulose, polyacrylamide, agarose and magnetite. The nature of the carrier can be either soluble or insoluble.

The formulations described herein may be administered in pharmaceutically acceptable compositions in some embodiments, which may routinely contain pharmaceutically acceptable concentrations of salt, buffering agents, preservatives, compatible carriers, adjuvants, emulsifiers, diluents, excipients, chelating agents, fillers, drying agents, antioxidants, antimicrobials, preservatives, binding agents, bulking agents, silicas, solubilizers, stabilizers and optionally other therapeutic ingredients, that may be used with the active compound. For example, if the formulation is a liquid, the carrier may be a solvent, partial solvent, or non-solvent, and may be aqueous or organically based. Non-limiting examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. The compositions of the invention may be formulated into preparations in solid, semi-solid, liquid or gaseous forms such as tablets, capsules, elixirs, powders, granules, ointments, solutions, depositories, inhalants or injectables, etc.

Preparations include sterile aqueous or nonaqueous formulations, suspensions and emulsions, such as creams, gels, lotions, and the like. In some cases, the preparations can be isotonic with the blood of the subject in certain embodiments. Non-limiting examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include, but are not limited to, alcoholic formulations, emulsions, or suspensions. Intravenous vehicles include fluid and nutrient replenishers, electrolyte replenishers (such as those based on Ringer's dextrose), and the like. Preservatives and other additives may also be present in some embodiments, such as, for example, antimicrobials, antioxidants, chelating agents, inert gases, and the like.

In some embodiments, a composition as described herein may be brought into association or contact with a suitable carrier, which may constitute one or more accessory ingredients. The final compositions may be prepared by any suitable technique, for example, by uniformly and intimately bringing a composition into association with a liquid carrier, a finely divided solid carrier, etc. optionally with one or more formulation ingredients as previously described.

The compositions as discussed herein, and optionally other therapeutics, may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. The term “pharmaceutically acceptable salts” includes salts of compositions described herein, prepared in combination with, for example, acids or bases.

Pharmaceutically acceptable salts can be prepared as alkaline metal salts, such as lithium, sodium, or potassium salts; or as alkaline earth salts, such as beryllium, magnesium, or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like. Also, such salts can be prepared as alkaline metal or alkaline earth salts, such as sodium, potassium, or calcium salts of the carboxylic acid group.

In another aspect, the present invention also provides any of the above-mentioned compositions in kits, optionally including instructions for use of the composition. In some cases, the kit can include a description of use of the compositions as discussed herein. The kit also can include instructions for use of a combination of two or more compositions. Instructions also may be provided for administering the compositions by any suitable technique as previously described, for example, orally, intravenously, pump or implantable delivery device, or via another known route of drug delivery.

The kits described herein may also contain one or more containers, which may contain compositions and other ingredients as previously described. The kits also may contain instructions for mixing, diluting, and/or administrating the compositions of the invention in some cases. The kits also can include other containers with one or more solvents, surfactants, preservatives, diluents, etc., as well as containers for mixing, diluting, or administering the components in a sample or to a subject in need of such treatment.

The compositions of the kit may be provided as any suitable form, for example, as a liquid. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

In still another aspect, the invention includes the promotion of one or more of the above-described embodiments, e.g., in vitro or in vivo, e.g., by administering, to a subject, compositions such as those described herein. As used herein, “promoted” includes all methods of doing business, including methods of education, scientific inquiry, academic research, industry activity including pharmaceutical industry activity, and any advertising or other promotional activity including written, oral and electronic communication of any form.

Each of the following is incorporated herein by reference in its entirety: U.S. Provisional Patent Application Ser. No. 62/713,022, filed Aug. 1, 2018, entitled “Anhydrous Dosage Forms of Aspirin Based on Deep Eutectic Solvents and Other Specialized Media”; U.S. Provisional Patent Application Ser. No. 62/778,949, filed Dec. 13, 2018, entitled “Systems and Methods for Delivery of Drugs and Other Substances Comprising Deep Eutectic Solvents”; U.S. Provisional Patent Application Ser. No. 62/778,954, filed Dec. 13, 2018, entitled “Methods and Systems Comprising Deep Eutectic Solvents for Poorly Soluble Drugs and Other Applications”; and U.S. Provisional Patent Application Ser. No. 62/791,110, filed Jan. 11, 2019, entitled “Methods and Systems Comprising Deep Eutectic Solvents for Poorly Soluble Drugs and Other Applications.”

The following examples are intended to illustrate certain embodiments of the present invention, but do not exemplify the full scope of the invention.

### Example 1

Examples of the invention are:
A liquid containing aspirin at a therapeutic concentration
A patch containing aspirin that delivers a prophylactic concentration
A cream containing aspirin the delivers a therapeutic concentration to painful tissue
A gel that delivers aspirin to the head at a therapeutic concentration for relief of headache

### Example 2

In this prophetic example, a subject is provided a liquid formulation which can be orally administered. The formulation contains aspirin dissolved in urea and choline chloride, in a ratio such that the formulation is liquid at room temperature.

### Example 3

In this prophetic example, a subject is provided a liquid formulation which can be orally administered. The formulation contains aspirin and caffeine dissolved in urea and choline chloride, in a ratio such that the formulation is liquid at room temperature.

### Example 4

In this prophetic example, a subject is provided a liquid formulation which can be orally administered. The formulation contains aspirin, caffeine, and acetaminophen dissolved in urea and choline chloride, in a ratio such that the formulation is liquid at room temperature.

### Example 5

In this example, an aspirin formulation was prepared using choline chloride and urea. In this example, choline chloride was heated in an oven at 65° C. for 10 hours. Then, 1 part choline chloride (by volume) was added to 2 parts urea in a beaker. The beaker was sealed with parafilm and the choline chloride and urea allowed to mix to form a deep eutectic solvent (DES). Next, the mixture was passed through a 4A 8-12 mesh molecular sieve, and stirred overnight, to form anhydrous DES.

50 mg/ml of aspirin was then added to the DES. However, only 1% of the aspirin was found to have hydrolyzed. The hydrolysis was determined using HPLC. Such a composition may be consumed orally.

As a comparison, if choline chloride and urea are mixed without heating the choline chloride using the same procedure as discussed above, then over 8% of the aspirin was found to hydrolyze. It is believed that heating the choline chloride removes water. Thus, this example illustrates that a formulation containing choline chloride, urea, and a small amount of water may be effective at producing a composition that does not significantly hydrolyze aspirin.

### Example 6

This example illustrates a method of making a formulation, in accordance with one embodiment of the invention. The formulation may or may not be anhydrous. First, a mixture of 1 part choline chloride to 2 parts urea by volume. That mixture was the mixed in a ratio of 1 part to 1 part propylene glycol. Next, 9 parts of that mixture was mixed with 1 part of citric acid. In one embodiment, this mixture was mixed with 25 mg/ml of loratadine. This would allow for the formulation of gel caps with, e.g., 10 mg or 20 mg loratidine per cap. The mixture may thus be administered, e.g., as a liquid, or in a gel cap. The effects of loratadine are expected to be much faster, as loratadine is normally relatively insoluble in water. In such a formulation, however, loratadine would be administered, e.g., to the stomach, in a “pre-dissolved” state.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition for oral delivery, comprising:
5 mol % to 20 mol % of one or more pharmaceutical agents comprising aspirin;
5 mol % to 95 mol % choline chloride;
5 mol % to 95 mol % urea; and
less than 1 mol % water,
wherein the composition is a liquid, and wherein the percentages of the pharmaceutical agents, choline chloride, urea, and water sum to at least 80 mol %.

2. The composition of claim 1, wherein the composition contains less than 0.1 mol % water.

3. The composition of any one of claims 1 or 2, wherein the composition contains less than 0.01 mol % water.

4. The composition of any one of claims 1-3, wherein the composition contains an undetectable amount of water.

5. The composition of any one of claims 1-4, wherein the composition is a liquid at room temperature.

6. The composition of any one of claims 1-5, wherein the composition has a melting point that is less than 25° C.

7. The composition of any one of claims 1-6, wherein the composition has a melting point that is less than 20° C.

8. The composition of any one of claims 1-7, wherein the composition has a melting point that is less than 10° C.

9. The composition of any one of claims 1-8, wherein the one or more pharmaceutical agents further comprises caffeine.

10. The composition of any one of claims 1-9, wherein the one or more pharmaceutical agents further comprises acetaminophen.

11. The composition of any one of claims 1-10, wherein the one or more pharmaceutical agents consist essentially of aspirin.

12. The composition of any one of claims 1-11, wherein the one or more pharmaceutical agents are present at between 5 mol % and 10 mol %.

13. The composition of any one of claims 1-12, wherein the choline chloride is present at between 25 mol % and 75 mol %.

14. The composition of any one of claims 1-13, wherein the urea is present at between 25 mol % and 75 mol %.

15. The composition of any one of claims 1-14, wherein the choline chloride is present at between 40 mol % and 60 mol %.

16. The composition of any one of claims 1-15, wherein the urea is present at between 40 mol % and 60 mol %.

17. The composition of any one of claims 1-16, wherein the choline chloride and the urea are a present within the composition at a ratio that causes the composition to have a melting point of less than 25° C.

18. The composition of any one of claims 1-17, wherein the choline chloride and the urea are a present within the composition at a ratio that causes the composition to have a melting point of less than 20° C.

19. The composition of any one of claims 1-18, wherein the percentages of pharmaceutical agents, choline chloride, urea, and water sum to at least 90 mol %.

20. The composition of any one of claims 1-19, wherein the percentages of pharmaceutical agents, choline chloride, urea, and water sum to at least 95 mol %.

21. The composition of any one of claims 1-20, wherein the composition consists essentially of one or more pharmaceutical agents, choline chloride, and urea.

22. The composition of any one of claims 1-21, wherein the composition consists essentially of aspirin, choline chloride, and urea.

23. The composition of any one of claims 1-21, wherein the composition consists essentially of aspirin, caffeine, choline chloride, and urea.

24. The composition of any one of claims 1-21, wherein the composition consists essentially of aspirin, caffeine, acetaminophen, choline chloride, and urea.

25. A method, comprising orally administering the composition of any one of claims 1-24 to a subject.

26. The method of claim 25, wherein the subject is human.

27. A method, comprising orally administering a composition to a subject, wherein the composition comprises:
5 mol % to 20 mol % of one or more pharmaceutical agents comprising aspirin;
5 mol % to 95 mol % choline chloride;
5 mol % to 95 mol % urea; and
less than 1 mol % water,
wherein the composition is a liquid, and wherein the percentages of pharmaceutical agents, choline chloride, urea, and water sum to at least 80 mol %.

28. The method of claim 27, wherein the composition contains less than 0.1 mol % water.

29. The method of any one of claim 27 or 28, wherein the composition is a liquid at room temperature.

30. The method of any one of claims 27-29, wherein the composition has a melting point that is less than 25° C.

31. The method of any one of claims 27-30, wherein the one or more pharmaceutical agents further comprises caffeine.

32. The method of any one of claims 27-31, wherein the one or more pharmaceutical agents further comprises acetaminophen.

33. The method of any one of claims 27-32, wherein the one or more pharmaceutical agents are present at between 5 mol % and 10 mol %.

34. The method of any one of claims 27-33, wherein the choline chloride is present at between 25 mol % and 75 mol %.

35. The method of any one of claims 27-34, wherein the urea is present at between 25 mol % and 75 mol %.

36. The method of any one of claims 27-35, wherein the choline chloride is present at between 40 mol % and 60 mol %.

37. The method of any one of claims 27-36, wherein the urea is present at between 40 mol % and 60 mol %.

38. The method of any one of claims 27-37, wherein the choline chloride and the urea are a present within the composition at a ratio that causes the composition to have a melting point of less than 25° C.

39. The method of any one of claims 27-38, wherein the percentages of pharmaceutical agents, choline chloride, urea, and water sum to at least 90 mol %.

40. The method of any one of claims 27-39, wherein the subject is human.

41. A method, comprising orally administering a composition to a subject, wherein the composition comprises:
5 mol % to 20 mol % of one or more pharmaceutical agents and/or beneficial substances;
5 mol % to 95 mol % choline chloride; and
5 mol % to 95 mol % urea,
wherein the composition is a liquid, and wherein the percentages of the pharmaceutical agents, the beneficial substances, choline chloride, and urea sum to at least 80 mol %.

42. The method of claim 41, wherein the composition contains less than 1 mol % water.

43. The method of any one of claim 41 or 42, wherein the composition is a liquid at room temperature.

44. The method of any one of claims 41-43, wherein the composition has a melting point that is less than 25° C.

45. The method of any one of claims 41-44, wherein the one or more pharmaceutical agents are present at between 5 mol % and 10 mol %.

46. The method of any one of claims 41-45, wherein the choline chloride is present at between 25 mol % and 75 mol %.

47. The method of any one of claims 41-46, wherein the urea is present at between 25 mol % and 75 mol %.

48. The method of any one of claims 41-47, wherein the choline chloride is present at between 40 mol % and 60 mol %.

49. The method of any one of claims 41-48, wherein the urea is present at between 40 mol % and 60 mol %.

50. The method of any one of claims 41-49, wherein the choline chloride and the urea are a present within the composition at a ratio that causes the composition to have a melting point of less than 25° C.

51. The method of any one of claims 41-50, wherein the one or more pharmaceutical agents comprises aspirin.

52. The method of any one of claims 41-51, wherein the one or more pharmaceutical agents comprises caffeine.

53. The method of any one of claims 41-52, wherein the one or more pharmaceutical agents comprises acetaminophen.

54. The method of any one of claims 41-53, wherein the one or more pharmaceutical agents has a solubility in water of less than 1000 mg/l.

55. The method of any one of claims 41-54, wherein the percentages of pharmaceutical agents, choline chloride, urea, and water sum to at least 90 mol %.

56. The method of any one of claims 41-55, wherein the subject is human.

57. A method, comprising orally administering a composition to a subject, wherein the composition comprises:
a deep eutectic solvent; and
one or more pharmaceutical agents and/or beneficial substances,
wherein the composition is a liquid, and wherein the percentages of the pharmaceutical agents, the beneficial substances, and the deep eutectic solvent sum to at least 80 mol %.

58. The method of claim 57, wherein the deep eutectic solvent comprises urea and choline chloride.

59. The method of any one of claim 57 or 58, wherein the deep eutectic solvent consists essentially of urea and choline chloride.

60. The method of any one of claims 57-59, wherein the deep eutectic solvent comprises at least 50 mol % of the composition.

61. The method of any one of claims 57-60, wherein the deep eutectic solvent comprises at least 80 mol % of the composition.

62. The method of any one of claims 57-61, wherein the deep eutectic solvent comprises at least 90 mol % of the composition.

63. The method of any one of claims 57-62, wherein the one or more pharmaceutical agents comprises aspirin.

64. The method of any one of claims 57-63, wherein the one or more pharmaceutical agents comprises caffeine.

65. The method of any one of claims 57-64, wherein the one or more pharmaceutical agents comprises acetaminophen.

66. The method of any one of claims 57-65, wherein the deep eutectic solvent has a melting point that is less than 25° C.

67. The method of any one of claims 57-66, wherein the one or more pharmaceutical agents has a solubility in water of less than 1000 mg/l.

68. The method of any one of claims 57-67, wherein the one or more pharmaceutical agents has a solubility in water of less than 100 mg/l.

69. The method of any one of claims 57-68, wherein the one or more pharmaceutical agents has a solubility in water of less than 10 mg/l.

70. The method of any one of claims 57-69, wherein the composition contains less than 1 mol % water.

71. A composition, comprising:
an emulsion comprising a first phase and a second phase substantially immiscible in the first phase, wherein the first phase comprises choline chloride, urea, and loratadine.

72. The composition of claim 71, wherein the composition is a gel.

73. The composition of claim 72, wherein the gel is contained within a capsule.

74. The composition of any one of claims 71-73, wherein the first phase contains less than 1 mol % water.

75. The composition of any one of claims 71-74, wherein the first phase contains less than 0.1 mol % water.

76. The composition of any one of claims 71-75, wherein the first phase contains an undetectable amount of water.

77. The composition of any one of claims 71-76, wherein the first phase is a liquid at room temperature.

78. The composition of any one of claims 71-77, wherein the first phase has a melting point that is less than 25° C.

79. The composition of any one of claims 71-78, wherein the first phase has a melting point that is less than 20° C.

80. The composition of any one of claims 71-79, wherein the first phase has a melting point that is less than 10° C.

81. The composition of any one of claims 71-80, wherein the choline chloride is present in the first phase at between 25 mol % and 75 mol % of the first phase.

82. The composition of any one of claims 71-81, wherein the urea is present in the first phase at between 25 mol % and 75 mol % of the first phase.

83. The composition of any one of claims 71-82, wherein the choline chloride is present in the first phase at between 40 mol % and 60 mol % of the first phase.

84. The composition of any one of claims 71-83, wherein the urea is present in the first phase at between 40 mol % and 60 mol % of the first phase.

85. The composition of any one of claims 71-84, wherein the choline chloride and the urea are a present within the first phase at a ratio that causes the first phase to have a melting point of less than 25° C.

86. The composition of any one of claims 71-85, wherein the choline chloride and the urea are a present within the first phase at a ratio that causes the first phase to have a melting point of less than 20° C.

87. The composition of any one of claims 71-86, wherein the percentages of loratadine, choline chloride, urea, and water in the first phase sum to at least 90 mol % of the first phase.

88. The composition of any one of claims 71-87, wherein the percentages of loratadine, choline chloride, urea, and water sum in the first phase to at least 95 mol % of the first phase.

89. The composition of any one of claims 71-88, wherein the first phase consists essentially of loratadine, choline chloride, and urea.

90. A method, comprising orally administering the composition of any one of claims 71-89 to a subject.

91. The method of claim 90, wherein the subject is human.

92. A transdermal patch, comprising:
a backing layer; and
an adhesive comprising choline chloride, urea, and one or more pharmaceutical agents.

93. The transdermal patch of claim 92, wherein the adhesive contains less than 1 mol % water.

94. The transdermal patch of any one of claim 92 or 93, wherein the adhesive contains less than 0.1 mol % water.

95. The transdermal patch of any one of claims 92-94, wherein the adhesive contains an undetectable amount of water.

96. The transdermal patch of any one of claims 92-95, wherein the urea and choline chloride form a eutectic mixture.

97. The transdermal patch of claim 96, wherein the eutectic mixture has a melting point of less than 25° C.

98. The transdermal patch of any one of claim 96 or 97, wherein the eutectic mixture has a melting point of less than 20° C.

99. The transdermal patch of any one of claims 96-98, wherein the eutectic mixture has a melting point of less than 10° C.

100. The transdermal patch of any one of claims 92-99, wherein the one or more pharmaceutical agents further comprises caffeine.

101. The transdermal patch of any one of claims 92-100, wherein the one or more pharmaceutical agents further comprises acetaminophen.

102. The transdermal patch of any one of claims 92-101, wherein the one or more pharmaceutical agents consist essentially of aspirin.

103. The transdermal patch of any one of claims 92-102, wherein the one or more pharmaceutical agents are present at between 5 mol % and 10 mol %.

104. The transdermal patch of any one of claims 92-103, wherein the choline chloride is present at between 25 mol % and 75 mol %.

105. The transdermal patch of any one of claims 92-104, wherein the urea is present at between 25 mol % and 75 mol %.

106. The transdermal patch of any one of claims 92-105, wherein the choline chloride is present at between 40 mol % and 60 mol %.

107. The transdermal patch of any one of claims 92-106, wherein the urea is present at between 40 mol % and 60 mol %.

108. A method, comprising applying the transdermal patch of any one of claims 92-107 to the skin of a subject.

109. The method of claim 108, wherein the subject is human.

110. A composition, comprising:
an emulsion comprising a first phase and a second phase substantially immiscible in the first phase, wherein the first phase comprises choline chloride, urea, and one or more pharmaceutical agents.

111. The composition of claim 110, wherein the composition is a cream.

112. The composition of any one of claim 110 or 111, wherein the composition is a lotion.

113. The composition of any one of claims 110-112, wherein the first phase contains less than 1 mol % water.

114. The composition of any one of claims 110-113, wherein the first phase contains less than 0.1 mol % water.

115. The composition of any one of claims 110-114, wherein the first phase contains an undetectable amount of water.

116. The composition of any one of claims 110-115, wherein the first phase is a liquid at room temperature.

117. The composition of any one of claims 110-116, wherein the first phase has a melting point that is less than 25° C.

118. The composition of any one of claims 110-117, wherein the first phase has a melting point that is less than 20° C.

119. The composition of any one of claims 110-118, wherein the first phase has a melting point that is less than 10° C.

120. The composition of any one of claims 110-119, wherein the one or more pharmaceutical agents further comprises caffeine.

121. The composition of any one of claims 110-120, wherein the one or more pharmaceutical agents further comprises acetaminophen.

122. The composition of any one of claims 110-121, wherein the one or more pharmaceutical agents consist essentially of aspirin.

123. The composition of any one of claims 110-122, wherein the one or more pharmaceutical agents are present in the first phase at between 5 mol % and 10 mol % of the first phase.

124. The composition of any one of claims 110-123, wherein the choline chloride is present in the first phase at between 25 mol % and 75 mol % of the first phase.

125. The composition of any one of claims 110-124, wherein the urea is present in the first phase at between 25 mol % and 75 mol % of the first phase.

126. The composition of any one of claims 110-125, wherein the choline chloride is present in the first phase at between 40 mol % and 60 mol % of the first phase.

127. The composition of any one of claims 110-126, wherein the urea is present in the first phase at between 40 mol % and 60 mol % of the first phase.

128. The composition of any one of claims 110-127, wherein the choline chloride and the urea are a present within the first phase at a ratio that causes the first phase to have a melting point of less than 25° C.

129. The composition of any one of claims 110-128, wherein the choline chloride and the urea are a present within the first phase at a ratio that causes the first phase to have a melting point of less than 20° C.

130. The composition of any one of claims 110-129, wherein the percentages of pharmaceutical agents, choline chloride, urea, and water in the first phase sum to at least 90 mol % of the first phase.

131. The composition of any one of claims 110-130, wherein the percentages of pharmaceutical agents, choline chloride, urea, and water sum in the first phase to at least 95 mol % of the first phase.

132. The composition of any one of claims 110-131, wherein the first phase consists essentially of one or more pharmaceutical agents, choline chloride, and urea.

133. The composition of any one of claims 110-132, wherein the first phase consists essentially of aspirin, choline chloride, and urea.

134. The composition of any one of claims 110-133, wherein the first phase consists essentially of aspirin, caffeine, choline chloride, and urea.

135. The composition of any one of claims 110-134, wherein the first phase consists essentially of aspirin, caffeine, acetaminophen, choline chloride, and urea.

136. A method, comprising orally administering the composition of any one of claims 110-135 to a subject.

137. The method of claim 136, wherein the subject is human.

138. A composition, comprising:
an emulsion comprising a first phase and a second phase substantially immiscible in the first phase, wherein the first phase comprises choline chloride, urea, and ketorolac.

139. The composition of claim 138, wherein the composition is a gel.

140. The composition of claim 139, wherein the gel is contained within a capsule.

141. The composition of any one of claims 138-140, wherein the first phase contains less than 1 mol % water.

142. The composition of any one of claims 138-141, wherein the first phase contains less than 0.1 mol % water.

143. The composition of any one of claims 138-142, wherein the first phase contains an undetectable amount of water.

144. The composition of any one of claims 138-143, wherein the first phase is a liquid at room temperature.

145. The composition of any one of claims 138-144, wherein the first phase has a melting point that is less than 25° C.

146. The composition of any one of claims 138-145, wherein the first phase has a melting point that is less than 20° C.

147. The composition of any one of claims 138-146, wherein the first phase has a melting point that is less than 10° C.

148. The composition of any one of claims 138-147, wherein the choline chloride is present in the first phase at between 25 mol % and 75 mol % of the first phase.

149. The composition of any one of claims 138-148, wherein the urea is present in the first phase at between 25 mol % and 75 mol % of the first phase.

150. The composition of any one of claims 138-149, wherein the choline chloride is present in the first phase at between 40 mol % and 60 mol % of the first phase.

151. The composition of any one of claims 138-150, wherein the urea is present in the first phase at between 40 mol % and 60 mol % of the first phase.

152. The composition of any one of claims 138-151, wherein the choline chloride and the urea are a present within the first phase at a ratio that causes the first phase to have a melting point of less than 25° C.

153. The composition of any one of claims 138-152, wherein the choline chloride and the urea are a present within the first phase at a ratio that causes the first phase to have a melting point of less than 20° C.

154. The composition of any one of claims 138-153, wherein the percentages of ketorolac, choline chloride, urea, and water in the first phase sum to at least 90 mol % of the first phase.

155. The composition of any one of claims 138-154, wherein the percentages of ketorolac, choline chloride, urea, and water sum in the first phase to at least 95 mol % of the first phase.

156. The composition of any one of claims 138-155, wherein the first phase consists essentially of ketorolac, choline chloride, and urea.

157. A method, comprising orally administering the composition of any one of claims 138-156 to a subject.

158. The method of claim 157, wherein the subject is human.
